(19) United States
(12) Patent Application Publication
(10) Pub. No.: US 2005/0000811 A1
(43) Pub. Date: Jan. 6, 2005

(54) Electrophoretically enhanced methods

(76) Inventor: Janos Luka, New Windsor, MD (US)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/839,714

(22) Filed: May 6, 2004

Related U.S. Application Data
(60) Provisional application No. 60/468,083, filed on May 6, 2003.

(51) Int. Cl. C12P 21/04; C08F 2/58; B01D 59/50

(52) U.S. Cl. 204/450; 204/600

(57) ABSTRACT

Electrophoretically enhanced methods are disclosed for carrying out binding and other reactions using pulsating and polarity reversing electric fields. The methods are exemplified by E3 ELISAs using free flow electrophoresis in multiwell plates and E3 Westerns using saturated matrices. The E3 methods are much faster than conventional methods and provide superior results. Reagents, buffers, devices and power supplied for carrying out E3 methods are disclosed.

This application is a continuation-in-part of and claims priority of U.S. provisional application No. 60/468,083 on “Electrophoretic Reaction Methods, and Compositions, Devices, and the Like Relating Thereto” of Janos Luka, Ph.D., filed 6 May 2003, which is herein incorporated by reference in its entirety.

### BACKGROUND

Except for unimolecular processes, chemical reactions universally involve the interaction of two or more molecules, and the rates and yields of most chemical reactions thus depend on the rate of the molecular contacts necessary for each of the steps in a reaction to occur and the frequency with which the contacts result in the step going forward. Thus, for instance, as the concentration of reactants in the vicinity of one another is increased—all other things remaining the same—the rate of contact between reactant molecules will increase and, consequently, the reaction between them will increase as well. Reactant concentrations and other factors that affect the speed, efficiency and yield of a reaction can be manipulated in many situations to achieve a satisfactory outcome. However, there also are many situations in which it is not possible to alter reaction conditions, such as concentrations, solvents or temperature, to achieve desired results.

This is particularly true for certain kinds of reactions that, by their nature always are partly dependent on circumstances such as inviolate starting material. Chemical analysis methods, for instance, must begin with a reaction that depends on the analyte and, if the analysis is to be quantitative, the reactions must in some way depend quantitatively on the amount of the analyte initially available in the starting material. Since the objective is to determine the amount of the analyte that is present, if any, it is not available as a parameter for optimizing the analytic reaction, except by increasing or decreasing the amount of the available sample used for the analysis. The former is useful only where there is more than enough sample to determine an analyte. The latter generally requires manipulating the sample to extract the analyte and processing the extract to obtain the analyte in the desired amount and concentration. All of the steps cause loss of analyte and introduce quantitative errors that limit their utility in analytical methods and/or limit the accuracy and usefulness of the analytical method. Even in the best case, however, the additional processing is inconvenient, takes time and adds cost. Often it is impractical. There may not be enough material, for instance, it may be too valuable, or it may be hard to obtain. Moreover, it is not possible in some cases to obtain a sufficient amount or a sufficient concentration of an analyte even with conventional methods of preprocessing.

Direct amplification would solve these problems; but, except for DNA, it is not possible to amplify molecules directly. And, even amplification of DNA by the polymerase chain reaction (“PCR”), while suitable for many purposes, has many limitations as well. For one, PCR is an exponential reaction and thus presents inherent problems for quantitative analysis. A slight, substrate-specific deviation from the expected efficiency of an exponential reaction, for instance, will produce large deviation in the final yield and, inaccuracy when the amount of starting material is extrapolated back from the yield. In addition, PCR can introduce and then amplify aberrant products that complicate or ruin reaction outcomes.

These problems are especially evident in biochemical analyses, especially in diagnostic and clinical analyses. Biological samples generally are available only in small amounts. The analytes typically are present only in some parts of the samples and not others. The amounts of significant analytes tend to be extremely low. Biological samples typically are complex and the analyte often is found together with other molecular species that are structurally similar but differ functionally and in analytical significance. Furthermore, analytes in biological samples often are subject to degradation, chemical modification, dissociation, masking and other artefact-producing reactions during the processes required to obtain, process, store and analyze the samples.

Given the importance of chemical reactions generally and of analytical reactions more particularly, especially, for instance, bioanalytical reactions, and the limitations on the ability to speed up, increase the efficiency and control the quality of many chemical processes, there is a need for improved methods, reagents, devices, systems and the like for increasing and/or optimizing the rate and/or efficiency and/or yield and/or quality and/or other qualities of chemical reaction processes.

This is true for reactions in which the reactants and the products are dissolved in solution. It also is true in which one or more of the reactants and/or one or more of the products is/are in a state other than dissolved in solution, including, for instance, a gel, a flocculent, or a solid state. In some ways, in fact, the need for methods to increase the rate, efficiency, yield and qualities, among others, of reactions is even greater for reactions in which one or more of the reactants, in particular, is immobilized, such as a reactant bound to a substrate or support, or trapped in a gel.

Many of the most advanced and promising techniques developed recently in chemical synthesis and analysis use surface-immobilized reactants and/or products of one type or another and, except for manufacturing processes, many of them also relate to very small amounts of material, complex sequential reactions, and simultaneous synthesis, reaction or analysis of very large numbers of individual chemical species in parallel.

For example, combinatorial chemistries used to synthesize large compound libraries rely on solid phase techniques. A typical combinatorial syntheses carried out on beads as the solid support might employ 6 successive rounds of synthesis involving 10 independent derivatizations to produce about one million different bead-immobilized compounds. Each compound will be attached to many different beads; but, each individual bead will have attached to it only one type of compound. The entire mixture then can be screened for a desired property all at once, in a small number of relatively large fractions, or in a greater number of smaller fractions. If the desired property is in evidence, the individual beads possessing the property then can be identified in the library by sub-division or capture or other techniques for this purpose. With the individual bead identified, the attached compound then can be determined, either by reading a tag incorporated during the synthesis reactions or by direct analysis, such as MALDI-TOF analysis or by another technique developed for this purpose.

Combinatorial synthetic methods have become an integral part of research and development efforts aimed at finding and refining new compounds useful for a wide range of applications. Illustrative in this regard is the widespread use of combinatorial synthesis in the development of new pharmaceutical agents. The large number of compounds produced by combinatorial chemistries have engendered a need for screening techniques that can be used—as a practical and economic matter—to subject millions of compounds to dozens of assays in a reasonable amount of time, using a reasonable amount of supplies and personnel, at a reasonable cost. A variety of high throughput screening (“HTS”) techniques have been developed for this purpose, along with the automated devices and systems to carry them out, and the information handling technology to acquire, store, process and display the resulting datasets.

Generally, HTS techniques to test compound libraries and for other types of testing are carried out using small volumes of reagents in multiwell plates. Most HTS assays rely on solid phase reactions in which an initiating reagent is immobilized on a surface, typically a flat surface of a microwell or of a “chip.” Typically, moreover, HTS methods rely on enzymic detection systems first developed for ELISAs. Indeed, many HTS procedures specifically are ELISAs. As described in greater detail below, however, despite the evident success of HTS methods developed to date, they often rely on assays that still are relatively slow and suffer from other problems, and there is a considerable need to improve their speed, sensitivity, efficiency, reproducibility, accuracy, specificity, yield and other properties, as described below illustratively for ELISAs in particular.

Arrays are another example of an enabling technology based on immobilized reagents and sequential solid phase reaction chemistries. Salient examples of array technologies include DNA chips and protein chips. Arrays also are being developed for carbohydrates, and they have been applied to developing novel inorganic compounds. All arrays involve a variety of solid phase reactions, in some cases, only as a matter of immobilizing the array compounds. In others, the arrays are made using sophisticated solid phase combinatorial chemistries. The use of arrays almost universally requires solid phase reaction chemistries as well. And these, more frequently then not, involve enzyme linked immunosorbant assays (“ELISAs”), similarly to HTS methods.

ELISAs originally were based exclusively on antigen-antibody interactions, and in a great many cases they still are; but, the core methodology of ELISA has been applied to other interactions, such as receptor ligand binding, and has used secondary interactions other than antibody-antibody binding, such as biotin-avidin and biotin-streptavidin interactions. At its heart, ELISAs and ELISA-like methods allow for easy visualization of results without radioactivity, based on the signal generating activity of an enzyme and a signal generating substrate analog. In ELISA assays a series of specific antibody-antigen and antibody-antibody interactions are used to bind enzyme molecules (e.g. horseradish peroxidase) to the bottom of a 96-well plate in such a way that the amount of enzyme is proportional to the amount of antigen in the system. The bound enzyme is incubated with a substrate analog that generates a product that can be detected and quantified, typically a colored product, a fluorescent product, a light-emitting product or light (among others). After a set incubation period under defined conditions the amount of product in the well is determined. The amount of the antigen (or other analyte) bound to the well then is calculated from the amount of the product generated by the bound enzyme.

In an indirect ELISA, fixed antigen is detected by a primary antibody, which is in turn detected by an enzyme-linked secondary antibody. In a capture or sandwich ELISA the antigen is “sandwiched” between a fixed capture antibody and an enzyme-linked detection antibody. In a competitive ELISA, known amounts of antigen are added back to a sandwich ELISA to compete for the detection antibody complex. Sensitivity of ELISAs are usually in the picogram per ml range using peroxidase/colorimetric systems, with a 10 to 100 fold increase using chemiluminescent detection systems.

Their specificity, reproducibility, sensitivity, and functional range (up to 5 logs with chemiluminescent detection) and their convenient plate format, which is easy to adapt to automated systems for high throughput screening have made standard ELISAs and ELISA-like methods key assay formats in immunology, cell biology, clinical testing, drug development and testing, toxicity determinations and high throughput screening, among others. They are a mainstay of clinical assays, biodefense, forensics, and biomedical research, as well as the methods discussed above. As a result the market for ELISAs worldwide is already quite large—billions of dollars—and growing rapidly.

Improvements in the performance of basic ELISAs and ELISA like methods are highly desirable and would have broad and positive effects. Unfortunately, basic ELISA technology has not been has not been significantly improved, except for the increase in sensitivity that was gained from the development of novel detection systems, such as fairly recently developed systems based on luminescence and chemiluminescence. ELISA technology would greatly benefit from improvements in a number of key areas, such as reducing the time for assays (presently several hours, typically 4 hours at minimum); reducing the amount of reagents required for many applications, particularly those involving scarce or expensive reagents; improving sensitivity which too often limits applicability of ELISAs; and reducing the background signal (noise) in many ELISAs, particularly those used with complex samples, such as serum, to name but a few needed improvements.

The benefits of improvements mentioned above would be broad. For example, a highly reliable 30 minute point of service ELISA that can be used to screen platelet for infectious agents could eliminate 24-48 hours of quarantined storage required prior to release for use, which would increase the functional shelf life of platelets by 20-30%. In addition, a rapid ELISA would be very advantageous for antigens with a short half life, such as post-translationally modified proteins, e.g. acetylated p53.

Many other analyte detection methods suffer some or all of the problems and shortcomings noted above for ELISAs. The same can be said for the speed, specificity, yield and other properties of chemical reactions.

Accordingly there is a need for improved assay methods and, further, for improved methods of directing chemical reactions.

### SUMMARY

It is therefore among certain objects of the present invention to provide electrophoretically enhanced methods for determining analytes, including especially methods that are faster than current methods. In certain particularly preferred embodiments in this regard the invention provides methods for determining analytes by binding reactions that are faster than other available methods and/or are more sensitive and/or provide a linear dose-response curve over a greater range than other available methods. In further particularly preferred embodiments in this regard the invention provides methods for determining analytes by binding reactions carried out on solid support. In especially preferred embodiments in this regard the invention provides electrophoretically enhanced ELISAs that are faster then conventional ELISAs for determining analytes. Among especially preferred embodiments in this regard are Electrophoretically Enhanced ELISAs for determining analytes in clinical tests. Also among especially preferred embodiments in this regard are Electrophoretically Enhanced ELISAs that are faster than conventional ELISAs for determining analytes in assays for High Throughput Screening. Further among especially preferred embodiments in this regard are Electrophoretically Enhanced hybridization assays that are faster than standard conventional hybridization assays.

Illustratively, and among a great many other things, the present invention provides a device, comprising, in one or in several parts: a first electrode, a second electrode, and a reaction vessel having: (a) an outside with an outside surface; (b) an inside with an inside surface; (c) a first opening and (d) a second opening, wherein the second opening is a semi-permeable ionically conductive membrane with an inside membrane surface that contacts the inside of the vessel and an outside membrane surface that contacts the outside of the vessel, wherein further, during operation: a first conductive medium is disposed in the device continuously so as to contact the first electrode, the second electrode and the outside membrane surface; a second conductive medium containing charged reactants or precursors thereto is disposed in the device between the inner membrane surface and the first conductive medium; the first electrode extends through the first opening into the inside of the reaction vessel and is disposed therein in contact with the first conductive medium but not in contact with the second conductive medium, the second electrode is disposed so as to contact the first conductive medium proximal to and in conductive contact with the outside membrane surface; wherein still further during operation voltage is applied to the first and second electrodes with net polarity that attracts the charged reactants to the membrane.

Devices as above further comprising any one or more of the following:
a first plurality of first electrodes and a second plurality of corresponding first openings, wherein during operation each first electrode of the first plurality extends through a corresponding first opening of the second plurality of corresponding first openings;
a third plurality of corresponding wells, wherein during operation each first electrode of the first plurality of electrodes extends through the corresponding first opening of the second plurality of corresponding first openings and into a corresponding well of said third plurality of corresponding wells.

Devices as above wherein further any one or combination of the following:
the vessel is a multiwell plate with semipermeable membrane bottom;
and/or the second conductive medium is more dense than the first conductive medium and initially is disposed in a layer on top of the inside membrane surface and below the first conductive medium;
and/or the semi-permeable membrane is a charged membrane or a neutral membrane; preferably a nylon, charged nylon, nitrocellulose or PVDF membrane, particularly preferably a charged nylon membrane or a PVDF membrane, particularly especially a PVDF membrane;
and/or the first conductive medium is characterized by low conductivity, pH and sufficient buffering capacity for the reactants to have net charge opposite that of the second electrode, preferably an organic buffer, a weak acid or a weak base, particularly preferably is a Tris-glycine, barbital or carbonate buffer;
and/or the first and second conductive media are the same except for the addition to the second conductive medium of a density agent that increases its density;
and/or a continuous voltage is applied during operation and/or a varying voltage is applied during operation and/or a switching voltage is applied during operation.

In additional aspects the invention also provides methods for enhancing reactions, comprising free flow electrophoresis of one or more reactants toward and/or onto a surface; methods for carrying out reactions, comprising concentrating one or more reactants onto a surface by free flow electrophoresis; and methods for carrying out reactions, comprising concentrating a first reactant near or onto a surface comprising a second reactant. In preferred embodiments the methods are solid phase assays, in particularly preferred embodiments bioanalytical solid phase assays, in related particularly preferred embodiments immunological solid phase assay; in particularly preferred embodiments in this regard enzyme linked immunosorbant assays and/or immunosorbant assays that use non-enzymatic detection. In additional preferred embodiments in this regard ligand binding solid phase assays, especially ELISA-like ligand binding assays, including those that use enzyme detection systems and those that use non-enzyme systems.

In further preferred embodiments in this regard the invention provides methods as set out above carried out in the foregoing devices of the invention as well.

In addition, the invention provides compositions for conducting free flow electrophoresis to concentrate a reactant on surface characterized by low conductivity and comprising Tris-glycine buffer of 50-150 mM Tris and correspondingly 300-900 mM glycine with pH 8.0 to 9.5; including in certain preferred embodiments also one or more of: 5 to 25 mM Histidine; 0.5 to 2.5% milk powder; 3%-30% density agent, preferably glycerol, sucrose or ficoll. Especially in this regard the invention provides compositions in accordance with the foregoing consisting in their buffering, density and blocking components essentially of 75 mM Tris, 450 mM glycine, 10 mM Histidine; pH 8.7, 2% milk powder and 20% glycerol or 75 mM Tris, 450 mM glycine or 75 mM Tris, 450 mM glycine, 10 mM Histidine; pH 8.7, 2% milk powder, 20% glycerol or 75 mM Tris, 450 mM glycine, 10 mM Histidine; pH 8.7, 2% milk powder or 75 mM Tris, 450 mM glycine, 10 mM Histidine; pH 8.7.

In other aspects of the invention certain of the preferred embodiments relate to systems, devices, methods, reagents and compositions of matter, inter alia, for performing electrophoretically enhanced processes and reactions on matrices, such as, for instance, in certain especially particularly preferred aspects and embodiments of the invention in this regard, Westerns.

Certain preferred aspects and embodiments of the invention relate in this regard to processes in which an electric field is applied across a membrane enhancing the formation complexes between reagents disposed in a medium surrounding the membrane or in a matrix contacting or adjacent to a face of the membrane and target substances immobilized on the membrane.

In a related aspect of the invention in this regard, in certain of the preferred embodiments the electric field is generated by the application across the membrane of a varying voltage, in particularly preferred embodiments a pulsing voltage, in especially particularly preferred embodiments a switching voltage, and in certain very especially particularly preferred embodiments a reversing pulse switching voltage.

In further related aspects of the invention in this regard, in certain highly especially preferred embodiments the voltage varies at relatively high frequency, in particularly preferred embodiments in this regard with a periodicity of 1 to 0.0001 second, especially particularly preferably 0.1 to 0.001 second, very especially particularly preferably 0.1 to 0.01 second.

Still further in this regard, in certain aspects and preferred embodiments the applied voltages are approximately square wave pulses of duration of 1 to 0.0001 second, preferably 0.1 to 0.001 second, particularly preferably 0.1 to 0.01 second.

In yet still further preferred embodiments in this regard, pulses of the foregoing preferred durations are applied with forward polarity more of the time then pulses with reverse polarity, and in certain very highly particularly preferred embodiments, pulses of approximately equal duration are applied, two thirds with forward polarity and one third with reverse polarity.

In particularly preferred aspects and embodiments for carrying out E3 Westerns in gels and with membranes of dimensions similar or the same as those commonly employed for conventional Westerns (10×10 cm, for example), preferred pulse programs have a pulse frequency of 100/sec, a pulse duration of about 10 milliseconds, an amplitude per pulse of about 10 volts, especially preferably with two of every three pulses having forward polarity and one of every three pulses having reverse polarity.

In yet another aspect the invention provide devices for carrying out electrophoretically enhanced reactions. The devices may be unitary but more often comprise several components. Preferred devices in this regard comprise a component for carrying out electrophoretically enhanced reactions and a power supply suitable for applying the electric field therefor. Preferred power supply components of the invention in this regard have current capacity of 0.1 to 10 mAmp/cm² of surface area of the electrodes, preferably, 0.3-7 mAmp/cm², particularly preferably 0.5-5 mAmp/cm², especially particularly preferably 0.8-3 mAmp/cm², yet still more especially particularly preferably 1-2 mAmp/cm².

Power supplies for E3 Westerns in this regard preferably have the capacity to provide 0-500 mAmp, and would handle between 100 and 200 mAmp of current, with an applied voltage, as noted above, switching between plus and minus 12 volts, using the E3 Western buffers described elsewhere herein.

In yet still further aspect and embodiments the invention provides the foregoing devices together with the buffers described above, and the application thereof to electrophoretically enhanced processes, especially preferably to E3 ELISAs and E3 Westerns.

In yet still further aspect and preferred embodiments the invention provides kits for carrying out electrophoretically enhanced reactions. The kits comprise one or more of the buffer components, buffers, reagents, detection reagents, standards, membranes, filters, sponges, ELISA plates, and/or other supplies and reagents used in carrying out electrophoretically enhanced processes, including, preferably, the buffers for E3 ELISA or E3 Westerns set forth above and elsewhere herein.

### BRIEF DESCRIPTION OF THE FIGURES

FIG. 1 is a schematic illustration of an exemplary device for carrying out certain types of electrophoretically enhanced assays as described herein.

FIG. 2 is a drawing that illustrates the disposition under running conditions of the electrodes, wells and buffers in an apparatus of the type depicted in FIG. 1. The lower electrode contacts filter paper dampened with running buffer. The capture antibody is immobilized on a charged hydrophobic nylon membrane that traps electrophoresis products, forms the bottom of the microplate wells and lies on top of the filter paper. Two buffers are used in the apparatus, Loading Buffer and Running Buffer. Loading Buffer is denser than Running Buffer. The membrane is covered by a layer of Loading Buffer. The dense layer of Loading Buffer is covered by Running Buffer. The upper electrode has pins that protrude into the wells of the plate, and extend just far enough to contact the Running Buffer in each well but not the Loading Buffer. In practice, by way of illustration with reference to a solid phase sandwich assay, after the capture antibody is immobilized on the membrane, the wells are washed and then partially filled with Running Buffer. The antigen is then applied in Loading Buffer and current is applied.

FIG. 3 is a schematic illustration of one possible mechanism for electrophoretic enhancement by way of reference to a sandwich ELISA.

Panel A shows capture antibody in solution above a membrane prior to immobilization. The antibody is in a buffer in which it is negatively charged. The membrane in the illustration is positively charged.

Panel B shows the capture antibody retained on the membrane through charge/hydrophobic interactions. Since all the antibody molecules are carrying a negative charge they are repulsed from one another and tend therefore to spread out on the membrane.

Panel C shows negatively charged antigen molecules prior to electrophoretically enhanced binding. The antigens are dissolved in a buffer designed to ensure that they are negatively charged.

Panel D shows antigens bound to the capture antibodies on the membrane following electrophoresis.

Panel E shows negatively charged detection antibody-signaling moiety complexes prior to electrophoresis.

Panel F shows the complex bound to antigen and antibody on the membrane in a stable tripartite immune complex.

FIG. 4 is a chart showing filter coating results obtained by an electrophoretic method in accordance with certain aspects of the invention compared with results obtained using weak vacuum.

FIG. 5 is a chart that illustrates increased sensitivity of electrophoretically enhanced assay methods compared to conventional methods in direct ELISAs of analytes in cell extracts. The chart shows results for E3 and conventional ELISA in detecting G3PDH in cell extracts. Except for the application of electrophoretic enhancement, the same standard direct ELISA protocol was used for both the conventional and the E3 assays. The determinations were carried out by coating the plates with a mammalian cell lysate, binding a polyclonal rabbit anti-G3PDH to G3PDH in the immobilized lysate, then binding an anti-rabbit IgG-peroxidase conjugate to bound anti-G3PDH in the wells. Finally, bound peroxidase conjugate was determined using the chromogenic substrate TMB. The comparison assays using the standard ELISA were carried out using standard polystyrene 96 well plates (Styrene) and standard 96 well filter bottom plates (ELISA).

FIG. 6 is a chart that illustrates the superior sensitivity of electrophoretically enhanced methods of the invention compared to the sensitivity of standard methods in a capture ELISA for detecting a viral capsid antigen in cell lysates. The sensitivity of an ELISA carried out using conventional and electrophoretically enhanced procedures was determined for the V3 Viral Capsid Antigen (VCA (V3)) of Epstein Barr Virus (EBV) using a biotin-labeled EBV VCA (V3)—specific antibody (anti-V3 (clone V16)) and a streptavidin-horseradish peroxidase conjugate. EBV VCA (V3) detection was determined by the membrane bound peroxidase activity that remained after the binding reactions were complete, and the membrane was washed. Peroxidase activity was measured calorimetrically as the amount of the chromogenic substrate TMB converted to colored product at each well location on the membrane using a scanning densitometer.

FIG. 7 is a chart that illustrates that the sensitivity of an electrophoretically enhanced direct ELISA in detecting an antigen in a complex mixture is superior to the sensitivity of the same ELISA carried out using conventional procedures. The experiments were carried out by first immobilizing on membranes known amounts of recombinant EBNA-1 antigen dissolved in 5% milk, 95% PBS. The immobilized antigen was detected by binding to it a mouse monoclonal anti-EBNA-1 antibody, washing, and then binding to the resultant complexes of bound antigen and monoclonal antibody a goat anti-mouse IgG-peroxidase conjugate. Enzyme conjugate bound to the antigen-antibody complex was visualized by the action of the peroxidase on the chromogenic substrate TMB. Enzyme activity was quantified by densitometry of colored product of the reaction on the membrane for each well using a scanning densitometer. Antigen detection was determined from the enzyme activity measured by this procedure for the reaction in each well.

FIG. 8 is a chart illustrating that the binding of antigen to capture antibody and of detection antibody to capture antibody-antigen complex occurs much more rapidly in ELISAs using electrophoretic enhancement then in the same ELISAs without electrophoretic enhancement. The data shown in the chart were obtained in a standard ELISA procedure carried out in the conventional manner or with electrophoretic enhancement, as described in greater detail elsewhere herein. In the conventional procedures (ELISA/10 and ELISA/60) the binding reactions for the capture antibodies and the detection antibodies were allowed to proceed either for 10 minutes each (ELISA/10) or for 60 minutes each (ELISA/60). In the electrophoretically enhanced procedure (E3) the binding reactions for the capture antibodies and the detection antibodies were allowed to proceed for 6 minutes each.

FIG. 9 is a chart showing the results obtained for the standard ELISA of modified DNA of Example 8.

FIG. 10 is a chart showing the results obtained for the Electrophoretically Enhanced ELISA of modified DNA of Example 8.

FIG. 11 is a diagram showing a set up for transferring material from a gel to a membrane in preparation for carrying out an E3 Western. (a) Lid of the transfer device. (b) Cathode. (c) Sponge(s). (d) Filter paper. (e) Gel. (f) Membrane. (g) Filter paper. (h) Blotting paper. (i) Anode. (j) Bottom of transfer device.

FIG. 12 is a diagram showing a set for antibody incubation for carrying out an E3 Western. (a) Lid of the device. (b) Cathode. (c) Sponge(s). (d) Filter paper. (e) Antibody saturated filter paper. (f) Membrane. (g) Filter paper. (h) Blotting paper. (i) Anode. (j) Bottom of transfer device.

### ABBREVIATIONS AND DEFINITIONS

Ab means antibody.
Ag means antigen.
Amp means ampere, the unit of electrical current.
mA means the same as mAmp.
mAmp means milliamp; i.e., milliampere; 10-3 ampere
cm means centimeter
cm² means centimeter square (i.e., a square one centimeter on each side).
E3 as used herein means Electrophoretically Enhanced and Electrophoretically Enhanced assays, in general and also is used in some instances to mean Electrophoretically Enhanced ELISAs, in particular.
E3 ELISAs means Electrophoretically Enhanced ELISAs.
E3 Westerns means Electrophoretically Enhanced Westerns.
EBV means Epstein Barr Virus.
EBV NA1 means Epstein Barr Virus Nuclear Antigen-1.
EIA is an acronym for Enzyme Immunoassay.
emf mean electromotive force: the force exerted by an electric field on a particle, such as a molecules; in other words the force exerted by a voltage on molecules and the like, which acts to move them and also may act to alter their internal motions and folding. Used herein in conjunction with the application of a voltage across electrodes and across a solution and/or a membrane and the like.
ELISA(s) is used herein generally to mean Enzyme Linked Immunosorbant Assay(s).
G3PDH means glyceraldehyde-3-phosphate dehydrogenase.
HTS means high throughput screening.
pI means isoelectric point, the pH at which a substance has net neutral charge.
PVDF means polyvinylidene fluoride.
TMB means tetramethylbenzidine.

### DESCRIPTION

By way of brief overview of the more detailed discussion and disclosure of the invention set forth below, in certain of its aspects and preferred embodiments thereof the invention herein described provides, among a great many other things, novel methods, compositions, devices and systems to enhance reactions using free flow electrophoresis, particularly reactions involving reactants in solution and a substrate, especially reactions in which a reactant in solution binds to a reactant on a solid support, particularly reactions in which a dissolved reactant binds with a reactant bound to a membrane, especially reactions to determine qualitatively or quantitatively one or more analytes, including particularly those in which either the analyte or the analyte binding moiety of an analyte-analyte binding moiety pair is bound to a membrane and the other is free in solution, especially particularly enzyme linked immunosorbant assays (ELISAs) and ELISA-like assays, particularly ELISAs, especially as to all of the immediately foregoing methods and the like in which free flow electrophoresis provides faster reactions and/or more stable binding complexes.

Free Solution Electrophoresis

Electrophoresis is the movement of charged particles by an electric field. When electrodes contact an electrolyte solution, positively charged particles migrate toward the cathode and negatively charged particles migrate to the anode. The rate of electrophoresis of a charged particle depends on the applied voltage (Field Strength), the particle's net charge (Net Charge on Particle) and the physicochemical properties of the particle and the solution that affect migration (Friction), according to the following equation:

$$Rate = \frac{( Field Strength ) ( Net Charge on Particle )}{( Friction )}$$

While theoretically correct, the equation is deceptively simple and it rarely can be used to predict or explain actual electrophoretic behavior. Nevertheless, certain types of electrophoresis has proven to be very robust and powerful bioseparation techniques. As a result, since it was first described by Tiselius (2) in the 1930s various electrophoresis procedures have developed into some of the most universal and frequently employed tools for bioanalytical separations.

Although electrophoresis was originally described for electrolytes in free solution, it is now performed almost exclusively in polymerized gel supports under denaturing conditions. Gels are used to control diffusion, convection and sources of unwanted motion. Presently, for instance, sodium dodecyl sulfate (SDS) polyacrylamide gel electrophoresis (PAGE) is by far the predominant electrophoretic method used to study and/or analyze proteins. SDS is a strong detergent that binds densely along the backbone of polypeptides. SDS protein binding gives proteins a net negative charge. Also repulsion between SDS molecules unfolds proteins. As a result, SDS converts complexly folded proteins into unfolded, linear polypeptides with constant charge to mass ratio. Consequently difference in the electrophoretic mobility of SDS-denatured proteins is a function of molecular friction and, since the linearized proteins are almost the same in this regard—except for length—SDS-PAGE is the technique of choice for separating proteins by molecular weight and for estimating their molecular weight, particularly if a gel matrix is used to control migration and provide sieving action (3).

In free solution electrophoresis, without SDS, most proteins have a similar net shape (globular) and, therefore, the contribution of molecular friction is relatively constant. Thus, under constant voltage and with no gel sieveing, the mobility of a protein is directly proportional to its net charge, not molecular weight. In addition, the migration of charged species in free solution electrophoresis is subject to diffusional effects and to the effect of heating by the applied electric field. These mixing effects distort migration due to the electric field and make it hard to control free solution migration.

Nor are these the only problems. When current flows in an aqueous electrolyte under the influence of an applied electric field, water is hydrolyzed at the electrodes. Hydrolysis at the anode produces acid and oxygen gas, while hydrolysis at the cathode produces hydroxide base and hydrogen gas, as follows:
At the anode: 2H₂O is electrolyzed to 4H⁺(acid)+O₂(gas)+4e⁻
At the cathode: 2H₂O is electrolyzed to 2OH⁻(base)+H₂ (g)
   Electrolysis at the electrodes consequently generates an acid front with dissolved oxygen gas and/or oxygen gas bubbles at the anode and an alkaline front with dissolved hydrogen gas and/or hydrogen gas bubbles at the cathode. The products and the conditions created by electrolysis around the electrodes in particular can cause significant oxidation or reduction of reactants, such as proteins, and bubbling at the electrodes can cause substantial physical movement of the buffer and of the components dissolved therein, disrupting their migration under the influence of the electric field.

These problems long have been seen as requiring a sieving matrix for practical applications of electrophoresis, and as something of an insurmountable barrier to using free solution electrophoresis for molecular separations. Thus there has not been much interest in the technique for quite some time, and there has been little interest in its use for quite some time, although it has been the object of some interest recently for separating DNAs (4).

Despite the long-standing view of free flow electrophoresis in this regard, the present invention overcomes these problems and provides methods, reagents, devices, systems and the like for using free solution electrophoresis to enhance reactions.

The invention provides, among other things, assays that: are extremely rapid compared to convention assays, have low levels of background noise and are more sensitive than comparable assays; are quantitative over a wider range than conventional assays, can be used easily even with complex matrices, in particular those that have been difficult to handle with conventional methods, such as serum samples and cell lysates, are highly reproducible and can readily be standardized, are relatively easy to automate, can be used to detect multiple analytes in a single well, require less reagent per assay than comparable conventional assays, readily can be carried out using many conventional, standard techniques and reagents, and lend themselves to novel applications beyond the capabilities of conventional assays.

In certain preferred embodiments of certain aspects of the invention, the methods, compositions, devices and systems and the like relate to solid phase assays. In certain particularly preferred embodiments in this regard, the invention relates to solid phase assays in which an analyte to be determined binds to and is thus indirectly immobilized by an analyte-specific analyte binding reagent. In these and other respects the invention, certain highly particularly preferred embodiments of the invention relate to enzyme linked immunosorbant assay (ELISA), particularly sandwich ELISAs, to ligand binding assays, particularly those in which ligands bind to immobilized ligand binding moieties, such as specific ligand-binding receptors. In these regards and others certain other highly particularly preferred embodiments of the invention relate to hybridization assays, especially solid phase hybridization assays in which one or more polynucleotides in a sample are hybridized to one or more probes immobilized and/or attached to a substrate.

In general electrophoretic enhancement in accordance with many aspects and preferred embodiments of the present invention relates to the application of an electric field (i.e., an electromotive force) to cause or direct motion of substances, particularly distinct molecular species and complexes that are reactants and/or products in chemical reactions and/or processes, thereby exert control over reactions and/or processes in which the substances are involved and/or which they otherwise influence.

In this respect, the invention can be applied in this respect to controlling a wide variety of reactions and processes ranging from analytical reactions designed to determine the presence of vanishingly small amounts of one or more particular substances in a sample to larger scale reactions for making significant amounts of desired products by chemical reaction processes.

In particularly preferred embodiments of certain aspects of the invention in this regard, the substances primarily are distinct molecules and/or macromolecular species. In particularly highly preferred embodiments in this regard the substances are distinct molecules and/or complexes of molecules that are charged. Especially particularly preferred in this regard are molecules and/or complexes thereof that are of biological origin or significance. Among particularly preferred embodiments, such molecules and/or complexes thereof are polynucleotides and/or complexes thereof, polypeptides and/or complexes thereof and carbohydrates and/or complexes thereof, including one or more of the foregoing. Among such polypeptides and complexes thereof are peptides and proteins that play a role naturally or that otherwise influence factors important to Apoptosis; p53 proteins and/or polypeptide and/or p53-related polypeptides and/or proteins and/or complexes comprising one or more of the foregoing p53 and/or p53-related polypeptides and/or proteins; peptides and/or proteins involved in generating, facilitating, preventing or ameliorating oxidative DNA damage; cytokines and cytokine-related peptides and/or proteins; viral antigens and/or complexes thereof and/or antibodies that recognize viral antigens and/or complexes thereof; and polypeptides and/or proteins that play a role in cell signaling, among others.

Further particularly preferred in this latter regard are molecules and/or complexes thereof that are of interest for the diagnosis, prognosis or treatment of disorder and/or disease and/or for the restoration, maintenance and/or prolongation of health. Especially highly particularly preferred in this regard are molecules and/or complexes thereof that are of clinical diagnostic interest and/or significance and/or are of interest and/or significance for clinical diagnostic research. By clinical diagnostic research in this regard is meant research relating to human health and/or disease, including research on any type of organism that may or actually is known to affect human health and/or disease, and any organism or biological phenomena that may provide useful information relating to human health and/or disease. Further by clinical diagnostic research is meant also all of the forgoing relating not only to human health and/or disease but also to health and/or disease of veterinary animals.

Among very highly particularly preferred aspects and embodiments of the invention relating to clinical and clinical diagnostic practice are molecules and/or complexes thereof useful and/or of significance and/or of interest for drug development, especially in this regard molecules and/or complexes thereof that are useful and/or are of significance and/or are of interest for high throughput screening to identify and/or validate and/or evaluate and/or develop candidate therapeutic agents and/or monitor their likely effects and/or efficacy and/or toxicity and/or side effects and/or safety for animal and/or human use.

Among further particularly preferred aspects and embodiments of the invention in this regard are molecules and/or complexes thereof of interest and/or significance for toxicity testing and/or research on toxicity and/or toxicity testing.

Among other particularly preferred aspects and embodiments of the invention in this regard are molecules and/or complexes thereof of interest and/or significance for environment monitoring and/or protection and/or remediation, including both naturally occurring molecules and/or complexes thereof.

Also particularly preferred in this regard are molecules or complexes thereof that are of interest for carrying out chemical reactions, including chemical reactions performed for research purposes and/or for development purposes and/or for scale-up purposes and/or for production purposes.

Further among particularly preferred aspects and embodiments of the invention in this regard are molecules and/or complexes thereof that are of significance and/or of interest in connection with nuclear and/or chemical and/or biological warfare. Especially highly particularly preferred in these particular regards are molecules and/or complexes thereof that are useful for monitoring to determine and identify the presence (and/or absence) of agents of nuclear and/or chemical and/or biological warfare.

Electrophoresis in Matrices and the Like

Notwithstanding the foregoing, however, the invention is not in any way limited to electrophoretic enhancement involving free flow electrophoresis exclusively. Indeed, to the contrary, the invention in certain aspects encompasses among its preferred embodiments a variety of methods and processes in which electrophoretic enhancement is achieved in whole or in part by electrophoresis not in free solution but in a matrix, such as a porous or fibrous matrix, such as a membrane or a paper, such as particularly, a filter paper or blotting paper. Electrophoretic enhancement in which electrophoresis is carried out in part in a matrix (filter paper) is illustrated by E3 Westerns of the invention, described in considerable detail herein below.

Electrophoretic Enhancement

The rate, extent, location, timing and other properties of reactions can be controlled and/or modulated and/or modified and/or influenced by electrophoretic methods in accordance with the present invention. By applying an electric field (voltage drop, voltage gradient, electromotive force) the motions of charged substances can be controlled and/or modulated and/or manipulated and/or modified and/or influenced so as, to name just a few, orient net motion in a particular direction, accelerate their motion, disorient their motion, slow their motion, bring them into close proximity, and/or spread them apart, and by doing so, for instance, accelerate and/or slow down and/or concentrate in a given location and/or spread out a reaction or reactions in which they are participants such as reactants, catalysts, co-factors, and/or products or that they influence.

In particular, in certain of its most highly preferred aspects and embodiments the invention relates in this regard to electrophoresis of substances in solution under the control of an applied voltage gradient, wherein in particularly preferred embodiments in this regard the substances are discreet molecules and/or complexes of molecular species in association with one another, especially, in still further particularly preferred embodiments in this regard, members of binding pairs, such as, in particularly especially preferred aspects and embodiments in this regard: antigens and/or antigen-specific antibodies and/or ligands and/or ligand-specific receptors and/or polynucleotides and/or complementary polynucleotides and/or complexes comprising any one or more of the foregoing and/or members of a binding pair derived from any one or more of the foregoing to name just a few.

In especially highly preferred embodiments of the invention in these and other regards, reactions are enhanced by controlling the electrophoresis of charged particles free in solution in buffer compositions designed in accordance with the invention as described elsewhere herein for free solution electrophoresis of molecules and/or molecular complexes of interest to control their participation in reactions, particularly in reactions carried out in small volumes. In particular, regarding free solution electrophoretic control of molecular motions to influence reactions, especially preferred are reactions taking place in or on a surface and/or reactions in which at least one reactant is immobilized and/or bound to a substrate and/or reactions that are carried out in a small volume. In these and other regards preferred substances are as set forth above.

In one unique and novel aspect of the present invention, free solution electrophoresis is utilized to move charged molecules rapidly in solution and to promote the formation of stable complexes, such as those of preferred embodiments mentioned above, including particularly in especially highly preferred embodiments in this regard complexes of polynucleotides hybridized to one another, complexes of immune reagents bound to one another (such as antigens and antibodies), complexes formed by the binding of ligands to ligand receptors, complexes formed by the binding of a lectin to lectin binding entities, and many other binding pairs, particularly binding pairs of molecules and/or complexes thereof.

In accordance with certain preferred embodiments of the invention further in these regards, an electric field is applied to an electrolyte-containing solution to control the motion of members of a binding pair so as to move them closer together and accelerate their binding one another and forming stable complexes. In particular, in some embodiments closer to one another, in some cases to concentrations much higher than those that can be achieved by standard methods. For instance, in using electrophoretic enhancement in accordance with the invention to enhance an immunoassay an electric field is applied to move antibodies close to antigens (or vice versa) thereby to accelerate and stabilize antigen/antibody complex formation.

Furthermore, under the conditions disclosed herein the complexes thus formed using electrophoretic enhancement methods in accordance with the invention not only form more rapidly but also are more stable than those formed by conventional methods, in particular immune complexes of antigen and antibody are more stable in this regard. Although the reasons for the unexpected stability of complexes are not understood fully, and any explanation therefore must remain hypothetical for now, the effect may result not from just one cause but from a combination of effects which may, in the case of immune complex for instance, include the impact of charge in promoting stabilization or “locking” of immune complexes; the rapid formation of a concentrated layer of antibody at the immune complex interface; promotion of “resonant movement” within the molecules and, once formed, the complexes; or other as yet unappreciated mechanisms.

Electrophoretic Enhancement Methods

In accordance with the invention herein disclosed electrophoresis is used to enhance reactions without substantial deleterious effects caused by anodic or cathodic electrohydrolysis, by heat conduction, electroosmotic flow, diffusion and other processes that engender mixing in fluids and that can disrupt the flow of charged particles towards an electrode in an electric field. The deleterious effects of these processes are overcome in accordance with certain preferred aspects and embodiments of the invention by new and unobvious compositions for carrying out free flow electrophoresis, in particular by new and unobvious buffer compositions. In particular, in certain of the especially preferred embodiments of the invention the deleterious effects are overcome and/or avoided by electrophoretic enhancement buffers for ELISAs and ELISA-like assays. Preferred buffer compositions also provide improved sensitivity, prevent leakage during electrophoresis, prevent damage to the antigen or antibody from the pH gradient formed at the electrodes, prevent oxidation or reduction by gases produced during electrophoresis, and prevent physical movement within the buffer from interfering with reactions enhancement.

Unlike buffers for other electrophoretic systems, buffers for electrophoretically enhanced reactions in accordance with the present invention in general, and those disclosed in the exemplary devices and methods herein described, are characterized by low conductivity and an absence of or very low concentrations of inorganic ions (consistent with low conductivity), appropriate pH and sufficient buffering capacity to ensure that reactants have the net charge required for migration to the membrane. Particularly, in preferred buffers in this regard, the conductivity and/or ionic strengths are sufficiently low that heat generation does not cause convection and consequent mixing of the buffer during electrophoresis. Further, preferred buffers in the same regard also are of sufficiently low conductivity to prevent, minimize or at least reduce gas production and the development of acidic or basic conditions at the electrodes to levels that will not substantially interfere with electrophoresis and will not damage or degrade reactants. Yet further, the conductivity and/or ionic strength of preferred buffers of the invention in this regard is such that currents that direct migration of reactants and thereby enhance a reaction also do not result in problematic heating and/or bubbling and/or gas generation and/or the building of high or low pH gradients and/or oxidative conditions and/or other conditions at or emanating from a cathode, an anode or both that interfere with, degrade or disrupt appreciable, markedly or substantially electrophoretic enhancement or that degrade, alter or otherwise deleteriously affect reactants in a way that degrades the enhancement or interferes with the desired reaction. Accordingly, among preferred buffering agents in accordance with the invention in this regard, are weak acid and weak base buffering agents, and, further in this regard organic buffering agents. Thus, as set forth in greater detail below, among particularly preferred buffers for electrophoretic reaction enhancement in accordance with certain aspects and preferred embodiments of the invention are Tris-glycine buffers and carbonate buffers.

In certain of the preferred embodiment in this regard, the pH of the buffers is above the isoelectric points of reactants and the buffers have sufficient buffering capacity to ensure that the reactants are negatively charged and will migrate toward the anode and thereby towards a membrane disposed between the reactants free in solution and the anode. As set forth in greater detail elsewhere herein for instance, in accordance with particularly preferred embodiments of the invention in various of its aspects, the buffers are used for electrophoretically enhanced ELISA and ELISA-like reactions, in which the analytes are one or more antigens and other reactants include one or more of antibodies, antibody-derived analyte-binding reagents, and secondary reactants that bind antibodies and/or antibody-derived reagents and/or antigens. Since antigens, antibodies and the foregoing antibody-derived molecules generally have an isoelectric point (“pI”) below approximately 8.0, the buffers for electrophoretic enhancement of ELISAs and ELISA-like assays with these reactants in accordance with the present invention have low conductivity and a pH above 8.0, preferably a pH of 8.3 to 9.7, particularly preferably a pH of 8.5 to 9.5, very particularly preferably a pH of 8.5 to 9.2, especially particularly preferably a pH of 8.6 to 9.1, very especially particularly preferably a pH of 8.6 to 8.8 or a pH of 8.9 to 9.1.

In especially preferred embodiments in these and other regards, the assays are ELISAs in which the analytes are small organic molecules or they are polypeptides and/or proteins and/or protein complexes and/or conjugates and the other reactants are polypeptides and/or proteins and/or protein complexes and/or conjugates, in particular antibodies and/or antibody derived polypeptides and/or proteins and/or conjugates or complexes of or derived from one or more polypeptides and/or proteins, generally having isoelectric points below the pH of the buffers for electrophoretic enhancement, generally below pH 8.3, and the enhancement buffers have a pH generally above pH 8.5, preferably a pH of 8.5 to 9.5, very particularly preferably a pH of 8.5 to 9.2, especially particularly preferably a pH of 8.6 to 9.1, very especially particularly preferably a pH of 8.6 to 8.8 or a pH of 8.9 to 9.1.

Among preferred buffers for electrophoretic enhancement in accordance with the invention in this regard are low conductivity, low ionic strength Tris-glycine buffers, of low conductivity, low ionic strength, preferably very low in inorganic ions, of pH 8.4 to 9.0, particularly preferably pH 8.5 to 8.9, very particularly preferably pH 8.6 to 8.8, especially particularly preferably pH 8.65 to pH 8.75, very especially particularly preferably approximately or exactly pH 8.7, preferably with sufficient buffering capacity to ensure that each reactant with a pI below the buffer pH by 0.2 (pH points) or more is negatively charged.

Among preferred buffering agents for use in the invention in this regard are organic buffering agents, such as Tris-glycine and barbitol.

Preferred concentrations of Tris-glycine in buffers in accordance with the immediately foregoing are 25 mM Tris, 150 mM glycine to 150 mM Tris, 900 mM glycine, particularly preferred is 50 mM Tris, 300 mM glycine to 100 mM Tris, 600 mM glycine, very particularly preferred is approximately or exactly 75 mM Tris, 450 mM glycine.

Also among preferred buffers for electrophoretic enhancement in accordance with the invention are low conductivity, low ionic strength carbonate buffers, preferably very low in inorganic ions, preferably with a pH of 8.6 to 9.4, particularly preferably a pH of 8.8 to 9.2, very particularly preferably a pH of 8.9 to 9.1, especially particularly preferably a pH of approximately or exactly 9.0.

Further relating to buffers, in certain preferred aspects and embodiments of the invention several related buffers are provided for performing electrophoretically enhanced reactions, particularly ELISAs and ELISA-like analyses, especially those involving proteins and polypeptides, particularly antigen determinations using antigen-specific antibodies. In this regard there are four types of preferred buffers, as discussed below.

(1) E3 Running Buffer

This buffer is used in accordance with preferred embodiments of the invention for electrophoretically enhanced reactions, particularly the reactions carried out in ELISA and ELISA-like assays. In accordance with the foregoing discussion of buffers of the invention, E3 Running Buffer has low conductivity, low ionic strength buffer, its pH is above the pI of the reactants, and it has sufficient buffering capacity to ensure that the reactants free in solution are negatively charged. In these regards, preferred embodiments of E3 Running Buffer compositions are as discussed generally for enhancement buffers above.

Among preferred buffering agents for use in the invention in this regard are organic buffering agents, such as Tris-glycine and barbital.

Among preferred buffers for electrophoretic enhancement in accordance with the invention in this regard are low conductivity, low ionic strength Tris-glycine buffers, of low conductivity, low ionic strength, preferably very low in inorganic ions, of pH 8.4 to 9.0, particularly preferably pH 8.5 to 8.9, very particularly preferably pH 8.6 to 8.8, especially particularly preferably pH 8.65 to pH 8.75, very especially particularly preferably approximately or exactly pH 8.7, preferably with sufficient buffering capacity to ensure that each reactant with a pI below the buffer pH by 0.2 (pH points) or more is negatively charged.

Preferred concentrations of Tris-glycine in buffers in accordance with the immediately foregoing are 25 mM Tris, 150 mM glycine to 150 mM Tris, 900 mM glycine, particularly preferred is 50 mM Tris, 300 mM glycine to 100 mM Tris, 600 mM glycine, very particularly preferred is approximately or exactly 75 mM Tris, 450 mM glycine.

Also among preferred buffers for electrophoretic enhancement in accordance with the invention are low conductivity, low ionic strength carbonate buffers, preferably very low in inorganic ions, preferably with a pH of 8.6 to 9.4, particularly preferably a pH of 8.8 to 9.2, very particularly preferably a pH of 8.9 to 9.1, especially particularly preferably a pH of approximately or exactly 9.0.

In addition to the foregoing preferred compositions, a very highly particularly preferred E3 Running Buffer in accordance with the invention in this regard is 75 mM Tris, 450 mM glycine, 10 mM Histidine; pH 8.7.

Typical Disposition of Running Buffer

In general, in preferred embodiments of the invention, E3 Running Buffer is in contact with both anode and cathode and (in reactions involving a membrane) the anode proximal side of the membrane. Initially, it also is in contact with the cathode-proximal side of the membrane. However, in preferred embodiments of the invention in this regard, a layer of E3 Loading Buffer containing reactants is layered over the membrane beneath the E3 Running Buffer, so that the Running Buffer is in contact with the Loading Buffer that lies above the membrane, but not the membrane itself in that portion covered by the Loading Buffer.

(2) E3 Loading Buffer
The composition of E3 Loading Buffer preferably is in accordance with the foregoing discussion of buffer compositions for electrophoretically enhanced reactions. Preferably it is a low conductivity, low ionic strength buffer, low in or lacking inorganic ions, with a pH above the pI of the reactants and sufficient buffering capacity to ensure that the reactants are negatively charged. E3 Loading Buffer in addition is denser than the Running Buffer in accordance with preferred embodiments of the invention in this regard, to facilitate underlaying the Running Buffer.

In preferred embodiments of the invention, E3 Loading Buffer has the same composition as E3 Running Buffer except that it also contains a density increasing agent. Among the preferred density increasing agents are high density reagents that also are non-conductive. Preferred density agents include, for instance, glycerol, ficoll and sucrose. In certain of the particularly preferred embodiments of the invention in this regard glycerol is used as the density increasing agent. Preferred concentrations of glycerol are 4% to 30%, particularly preferably 5% to 25%, very particularly preferably 10% to 25%, especially particularly preferably 15% to 25%, very especially particularly preferably 17% to 23%, very highly especially particularly preferably 19% to 21%, further especially approximately or exactly 20%.

Among preferred buffering agents for use in the invention in this regard are organic buffering agents, such as Tris-glycine and barbital.

Among preferred buffers for electrophoretic enhancement in accordance with the invention in this regard are low conductivity, low ionic strength Tris-glycine buffers, of low conductivity, low ionic strength, preferably very low in inorganic ions, of pH 8.4 to 9.0, particularly preferably pH 8.5 to 8.9, very particularly preferably pH 8.6 to 8.8, especially particularly preferably pH 8.65 to pH 8.75, very especially particularly preferably approximately or exactly pH 8.7, preferably with sufficient buffering capacity to ensure that each reactant with a pI below the buffer pH by 0.2 (pH points) or more is negatively charged.

Preferred concentrations of Tris-glycine in buffers in accordance with the immediately foregoing are 25 mM Tris, 150 mM glycine to 150 mM Tris, 900 mM glycine, particularly preferred is 50 mM Tris, 300 mM glycine to 100 mM Tris, 600 mM glycine, very particularly preferred is approximately or exactly 75 mM Tris, 450 mM glycine.

Also among preferred buffers for electrophoretic enhancement in accordance with the invention are low conductivity, low ionic strength carbonate buffers, preferably very low in inorganic ions, preferably with a pH of 8.6 to 9.4, particularly preferably a pH of 8.8 to 9.2, very particularly preferably a pH of 8.9 to 9.1, especially particularly preferably a pH of approximately or exactly 9.0.

Also preferred in certain aspect of the invention as to all of the foregoing preferred embodiments relating to Loading Buffer, are Loading Buffers that also prevent non-specific binding. Loading Buffers preferred in this regard are any of the foregoing preferred Loading Buffers containing a blocking agent in accordance with the Blocking Buffers described in the discussion of Blocking Buffers below.

A very highly particularly preferred E3 Loading Buffer is 75 mM Tris, 450 mM glycine, 10 mM Histidine; pH 8.7, 2% milk powder, 20% glycerol.

Loading Buffers to Prevent Membrane Leakage

In another aspect of the invention pertinent to buffers for electrophoretic enhancement in general and E3 Loading Buffers in particular, preferred buffers prevent leakage of buffers and reactants through membranes as follows.

Filter bottom plates can leak. Different plates have different propensities to leakage. Custom plates with small pore nylon membranes generally do not leak under the conditions typically used for electrophoretically enhanced assays in accordance with the invention. When prefabricated plates are used for electrophoretically enhanced reactions, however, leakage can occur through the membrane that can be a significant problem. The leakage often occurs specifically through relatively large pores in the membranes used for standard filter bottom well plates available commercially.

Leakage and the problems it causes can be prevented by Loading Buffers in accordance with certain of the particularly preferred embodiments of various aspects of the invention. These buffers provide an inexpensive and generally applicable solution to leakage problems that can occur when carrying out electrophoretic enhancement methods of the invention, particularly in standard filter bottom plates.

Preferred EE Loading Buffers to prevent leakage in this regard are much as described above for preferred buffers in general and for Loading Buffers in particular, except that buffers to prevent leakage are denser. The desired Loading Buffer density to prevent membrane leakage can be achieved by addition of a density agent to a Loading Buffer composition as described above. The density agent, as described for Loading Buffer above can be glycerol, ficoll or sucrose. The density of buffers in this regard can be in accordance with this aspect of the invention, sufficiently dense to prevent leakage. The densities should be equivalent to approximately 20% glycerol; but, less can be used if it prevents leakage effectively, and it may be necessary to use higher concentrations if 20% glycerol does not accomplish the goal. Particularly preferred are such dense loading buffers that also block non-specific binding to the membrane. An especially preferred EE Loading Buffer for ELISAs in this regard is 75 mM Tris, 450 mM glycine, 10 mM Histidine; pH 8.7, 2% milk powder and 20% glycerol.

Typical Disposition of Loading Buffer

In general, in preferred embodiments, reactants for electrophoretic transport are dissolved in Loading Buffer, and the Loading Buffer is layered onto the cathode-proximal face of a membrane, underneath the Running Buffer and disposed also so that it does not directly come into contact with the cathode. Thus disposed, the Loading Buffer forms a layer between the membrane and the Running Buffer that does not come into contact with the cathode.

(3) E3 Blocking Buffer

E3 Blocking Buffer contains blocking formulations designed to reduce non-specific binding to the membrane that can give rise to background signals. In addition to the foregoing buffer characteristics, particularly low conductivity, low ionic strength and appropriate pH relative to pertinent pIs, blocking also contains blocking agent that prevents non-specific binding and reduces background noise of reactions. Preferred E3 Blocking Buffer has low conductivity for free solution electrophoresis and provides better blocking of non-specific binding to nylon membranes than other standard blocking solution formulations. Particularly preferred E3 Blocking Buffers in accordance with the invention in this regard have the same composition as the Running Buffer except that it also contains a suitable blocking agent in a concentration effective for blocking non-specific binding and/or reducing and/or minimizing background signals and noise that reduce sensitivity and/or accuracy and/or signal to noise ratio and/or yield and/or other aspects of performance of the electrophoretically enhanced reaction.

Among preferred buffering agents for use in the invention in this regard are organic buffering agents, such as Tris-glycine and barbital.

Among preferred buffers for electrophoretic enhancement in accordance with the invention in this regard are low conductivity, low ionic strength Tris-glycine buffers, of low conductivity, low ionic strength, preferably very low in inorganic ions, of pH 8.4 to 9.0, particularly preferably pH 8.5 to 8.9, very particularly preferably pH 8.6 to 8.8, especially particularly preferably pH 8.65 to pH 8.75, very especially particularly preferably approximately or exactly pH 8.7, preferably with sufficient buffering capacity to ensure that each reactant with a pI below the buffer pH by 0.2 (pH points) or more is negatively charged.

Preferred concentrations of Tris-glycine in buffers in accordance with the immediately foregoing are 25 mM Tris, 150 mM glycine to 150 mM Tris, 900 mM glycine, particularly preferred is 50 mM Tris, 300 mM glycine to 100 mM Tris, 600 mM glycine, very particularly preferred is approximately or exactly 75 mM Tris, 450 mM glycine.

Also among preferred buffers for electrophoretic enhancement in accordance with the invention are low conductivity, low ionic strength carbonate buffers, preferably very low in inorganic ions, preferably with a pH of 8.6 to 9.4, particularly preferably a pH of 8.8 to 9.2, very particularly preferably a pH of 8.9 to 9.1, especially particularly preferably a pH of approximately or exactly 9.0.

A very highly particularly preferred E3 Blocking Buffer in accordance with the invention in this regard is 75 mM Tris, 450 mM glycine, 10 mM Histidine; pH 8.7, 2% milk powder.

(4) E3 Coating Buffer

Coating Buffer is for electrophoretically enhanced immobilization of substances onto membranes and other supports in accordance with the invention.

Among preferred buffering agents for use in the invention in this regard are organic buffering agents, such as Tris-glycine and barbital.

Among preferred buffers for electrophoretic enhancement in accordance with the invention in this regard are low conductivity, low ionic strength Tris-glycine buffers, of low conductivity, low ionic strength, preferably very low in inorganic ions, of pH 8.4 to 9.0, particularly preferably pH 8.5 to 8.9, very particularly preferably pH 8.6 to 8.8, especially particularly preferably pH 8.65 to pH 8.75, very especially particularly preferably approximately or exactly pH 8.7, preferably with sufficient buffering capacity to ensure that each reactant with a pI below the buffer pH by 0.2 (pH points) or more is negatively charged.

Preferred concentrations of Tris-glycine in buffers in accordance with the immediately foregoing are 25 mM Tris, 150 mM glycine to 150 mM Tris, 900 mM glycine, particularly preferred is 50 mM Tris, 300 mM glycine to 100 mM Tris, 600 mM glycine, very particularly preferred is approximately or exactly 75 mM Tris, 450 mM glycine.

Also among preferred buffers for electrophoretic enhancement in accordance with the invention are low conductivity, low ionic strength carbonate buffers, preferably very low in inorganic ions, preferably with a pH of 8.6 to 9.4, particularly preferably a pH of 8.8 to 9.2, very particularly preferably a pH of 8.9 to 9.1, especially particularly preferably a pH of approximately or exactly 9.0.

A very highly particularly preferred E3 Coating Buffer in accordance with the invention in this regard is 75 mM Tris, 450 mM glycine.

Membranes

The present invention in certain preferred aspects and embodiments relates to reactions performed on a solid support in which reactants are transported from solution to the membrane by free flow electrophoresis. Membranes suitable for use in the present invention include semi-permeable membranes, membranes that, particularly when wetted with suitable buffer, particularly a preferred buffer of the invention, including in particular the preferred E3 Running Buffers and/or E3 Loading Buffers and/or E3 Blocking Buffers and/or E3 Coating Buffers described herein above and those that are permeable to an ion current, particularly those that also bind reactants, particularly those that have a high capacity for the reactants, and are suitable for carrying out solid phase reactions, particularly, for instance, those that are inert with respect to the reactants and products of desired reactions, and those that have the desired binding properties for reactions components, in some cases irreversible binding being more desirable and in others reversible binding being more desired, in all these respects and others especially those reactions of the preferred embodiments described, such as especially the ELISAs and ELISA-like reactions discussed above.

Preferred membranes also are inert with respect to the buffers, reactants, products and other species involved in the application. Preferred membranes furthermore have high capacity for binding the species and complexes involved in the reactions of interest. Suitable membranes generally and preferably are porous.

Both charged and neutral membranes are suitable for use in the invention. Positively charged membranes are useful for anionic reactants that migrate to the anode under conditions of electrophoretic enhancement. Negatively charged membranes may be useful in much the same way for cationic reactants that migrate to the cathode. In general neutral membranes are preferred, particularly for ELISAs for determining analytes that are antigens using reactants that are antibodies or derived from antibodies.

Among membranes suitable for use in the present invention in this regard are nitrocellulose membranes, charged nylon membranes, neutral nylon membranes and PVDF membranes, including those available from commercial suppliers. Nylon and PVDF are particularly preferred. PVDF is very particularly preferred.

Among preferred membranes further in this regard are membranes provided by commercial vendors attached to the bottom of titer plates and forming the bottom of the microtiter well thereof. Further preferred in this regard are membranes attached to the underside of 96 well bottomless plates using, for example, sonic welding. This procedure forms a watertight seal to prevent leakage and cross-talk between adjacent wells. Most commonly, the weld is permanent and the filter may not then be peeled off. However, the temperature during welding may be controlled so that the bond between filter and plastic is watertight but allows for the filter to be manually separated. Membrane removal allows for a variety of alternate detection methods to be used, e.g. membrane-based colorimetric or chemiluminescent methods, as well as only partial plate use. Strip wells, containing 6 wells per strip are also available. Membranes are usually supported with a second lower layer of, for example, cellulose, and beneath that is a standard filter layer, usually with a small hole for easy removal of reagents under vacuum.

Features of the different commercially available membranes we considered were support, composition, charge, hydrophobicity, pore size, and binding capacity. A positively charged hydrophobic 0.45 micron nylon membrane gave optimal results in qualitative preliminary binding assays using a membrane based calorimetric detection system.

A major obstacle with the prefabricated plates was wicking of solution out of the wells during electrophoresis, that would occur randomly across the well. This problem appeared to be due to the presence of the hole in the lowest filter and whether all layers of membrane and filter were in direct contact. When dry, the membrane and filter have the same surface area, but once wet, the lowest filter layer increased in surface area that caused buckling. The problem of leakage was ultimately solved using modifications to the loading buffer.

Running Procedures

The voltage, current, power and other parameters of electrophoresis depend on a variety of factors, including but not limited to the buffer compositions, the nature of the sample, the nature of the reactants, the reaction to be enhanced, the products produced by the reactions and how ultimately they will be treated, the surface on which the electrophoretically enhanced reaction will take place, the apparatus in which the electrophoresis is being conducted and a variety of other factors.

The most important limitations on running conditions for electrophoretic enhancement are imposed by the current heating and concomitant mixing deleterious to migration of the reactants, and the deleterious effect of electrode reactions that generate acid, bass, hydrogen gas, oxygen gas, bubbles that cause mixing, and heat, among others. As discussed above, generation of these effects depends on the buffer among other important considerations. Thus, the running conditions should be matched to the buffers being used. The effects also depend on the type of support being used, and running conditions thus should be adjusted to match the particular membrane in use, as well as the buffers.

In general, desirably, running conditions ensure adequate migration of reactants to the support while also avoiding these harmful effects. Preferred conditions for electrophoresis include current and power regulated electrophoresis, particularly current regulated electrophoresis.

Among preferred current regulated conditions are currents of 0.15 to 2.50 mAmp per cm² of surface area (the surface area being in the plane perpendicular to the current flow; i.e., the plane through which the current is flowing. For instance, the bottom of a single well in a standard 96 well microtiter plate with circular wells has a diameter of about 0.8 cm and an area of approximately 0.6 cm². Among preferred running conditions in accordance with the present standard using 96 well filter bottom plates is current regulated electrophoresis at 0.1 to 1.5 mAmp per conducting well (9.6 mAmp to 144 mAmp for a microtiter plate in which all the wells contain buffer and are conducting current).

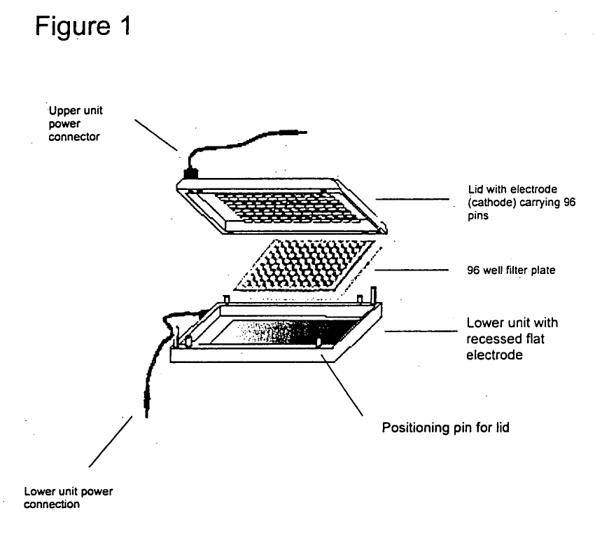

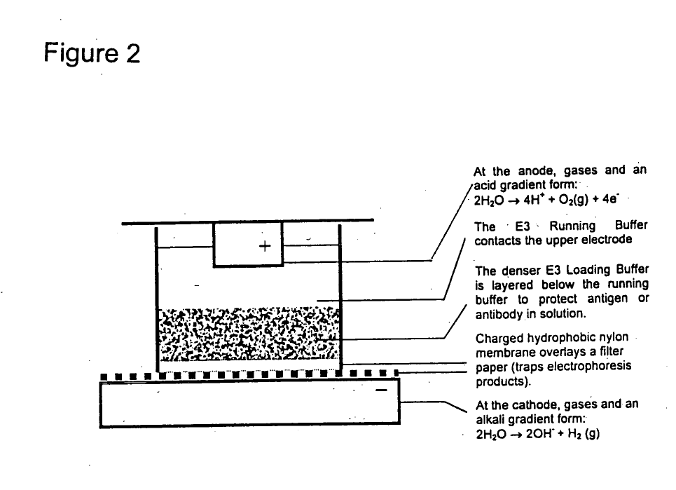

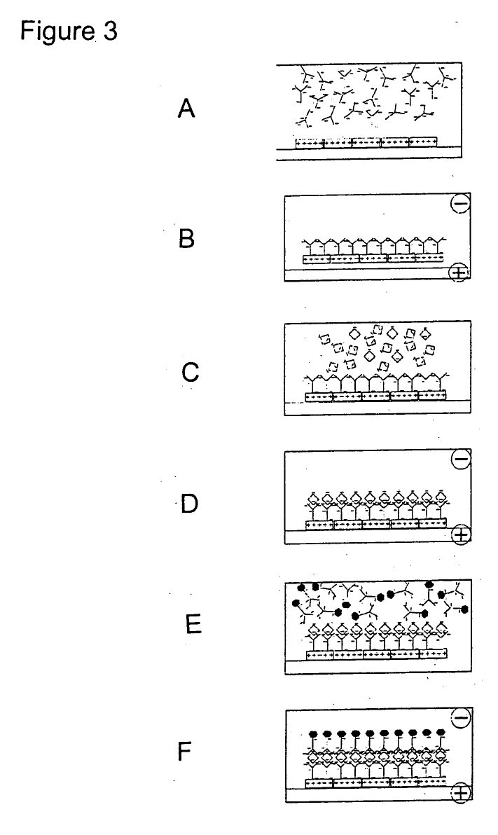

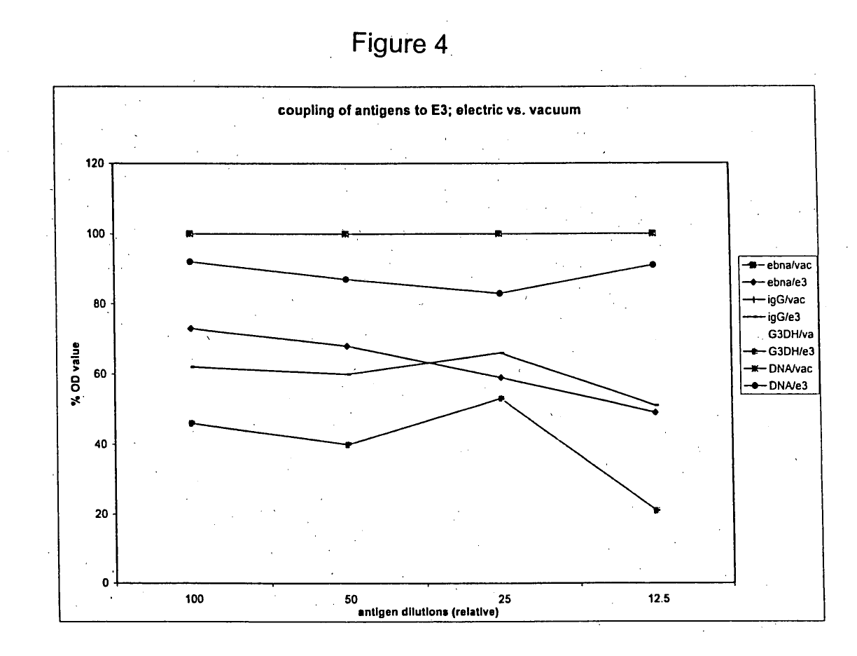

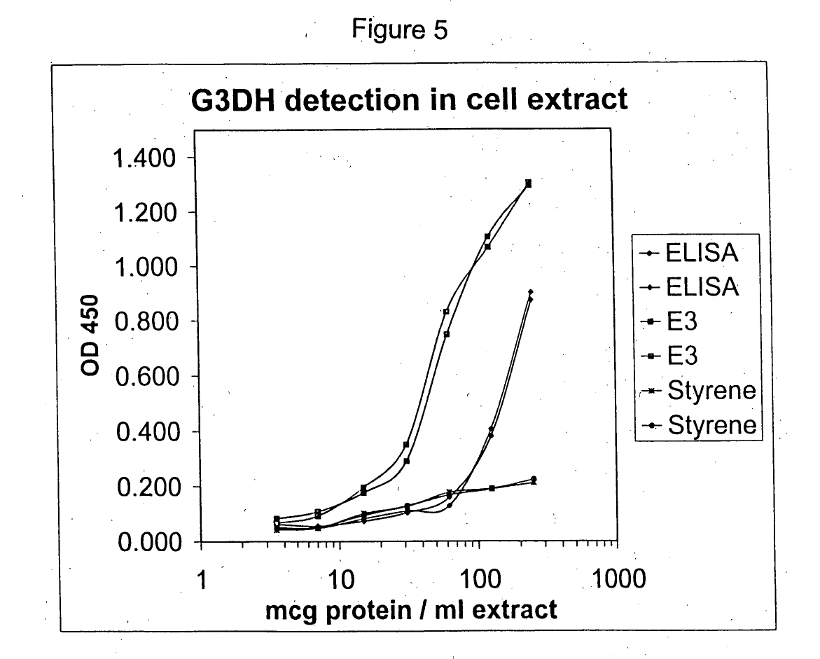

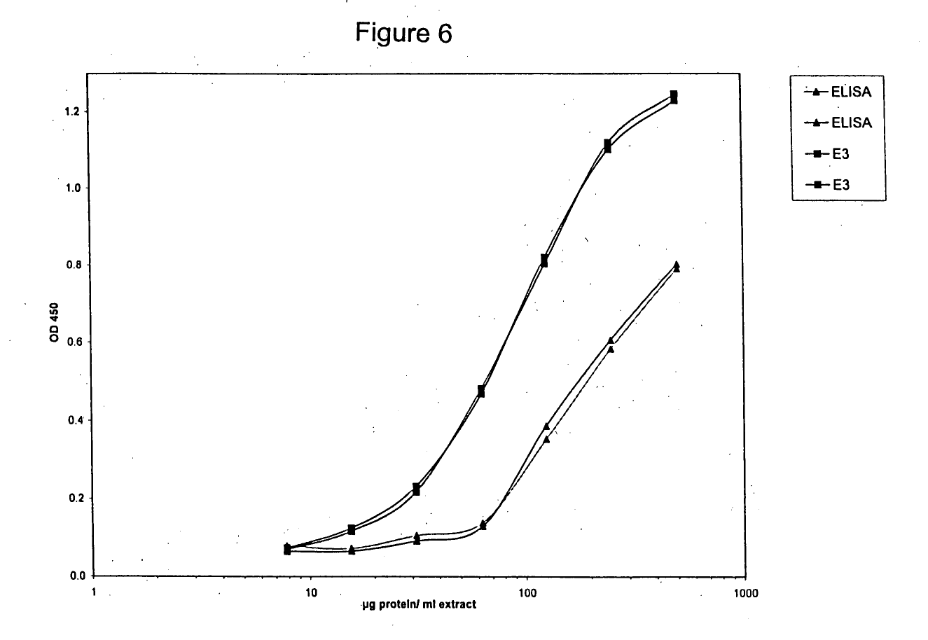

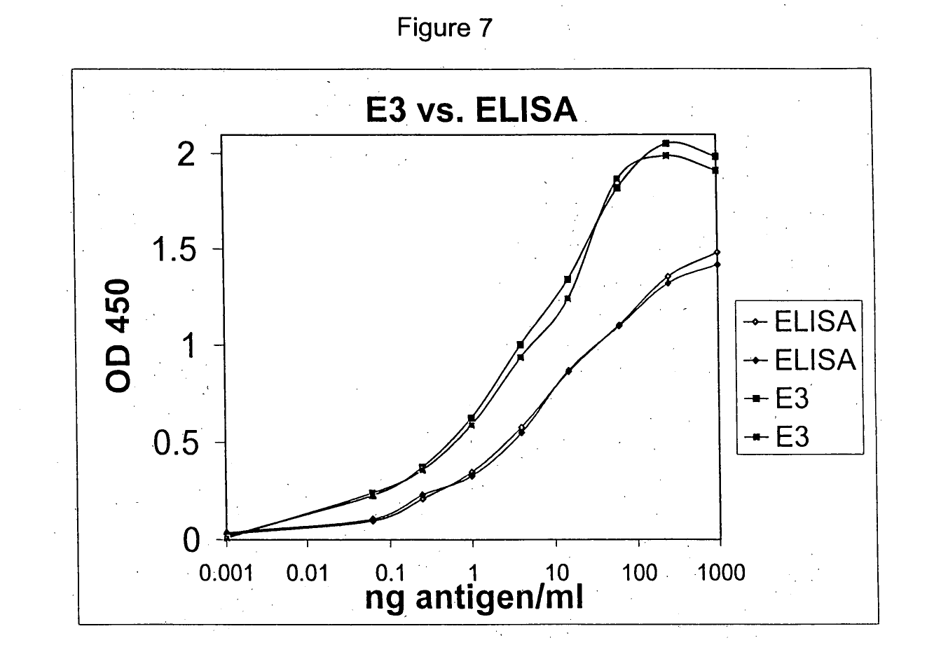

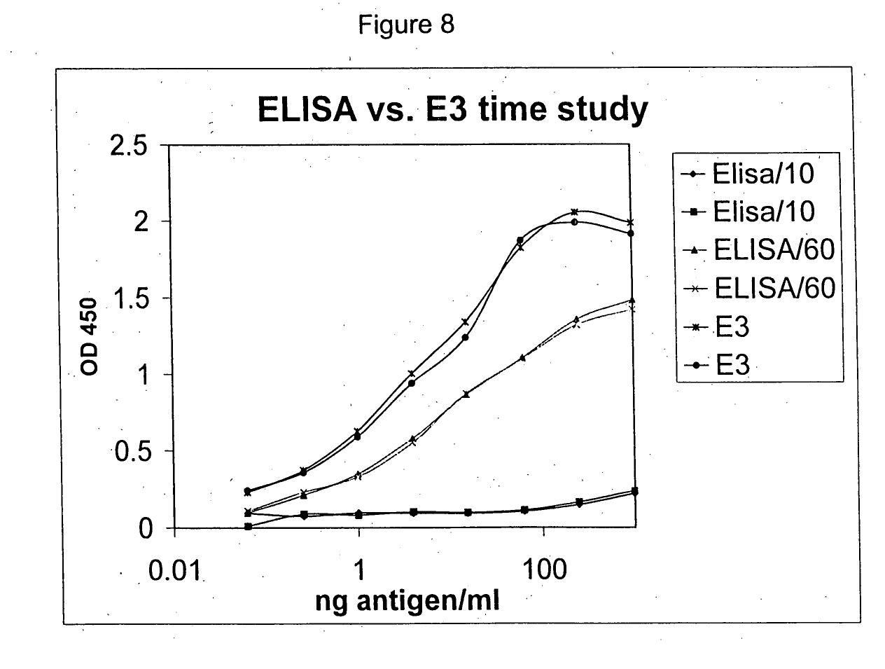

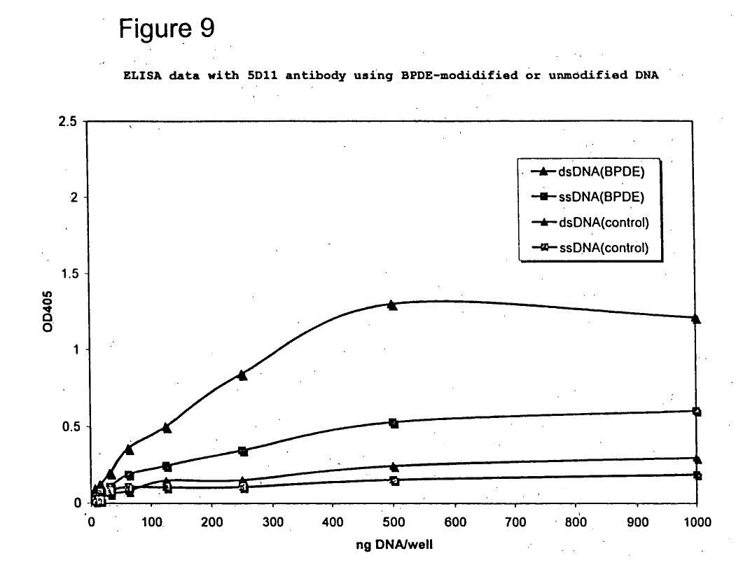

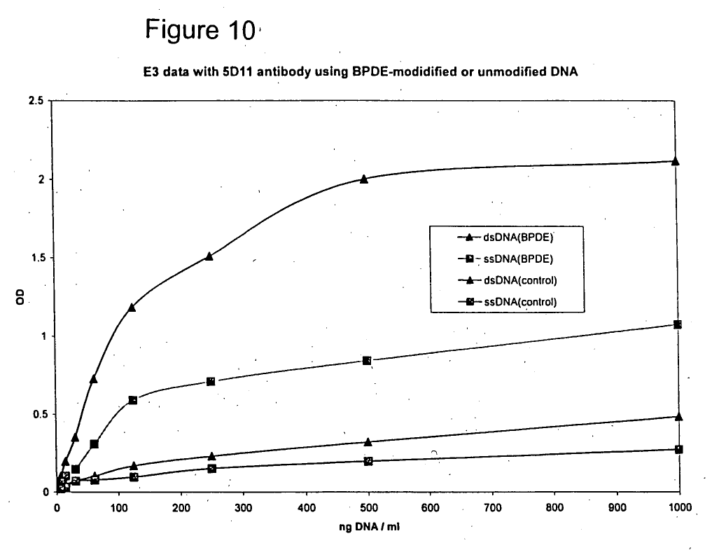

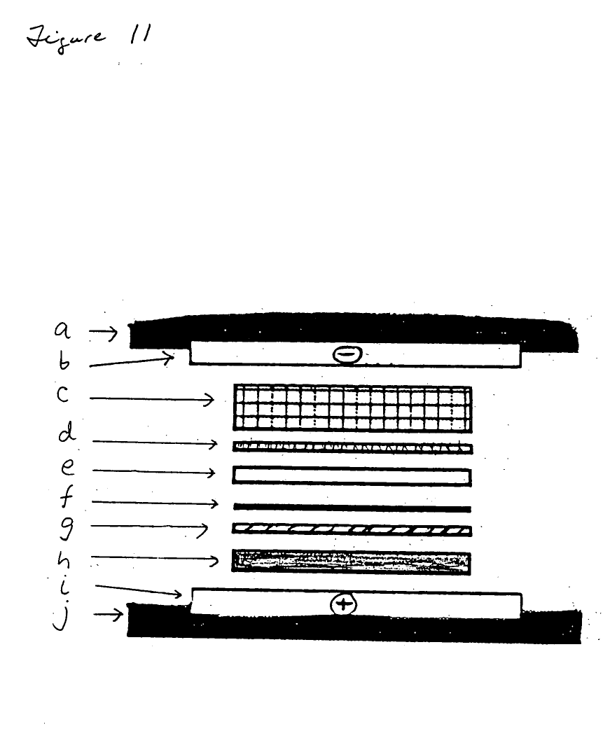

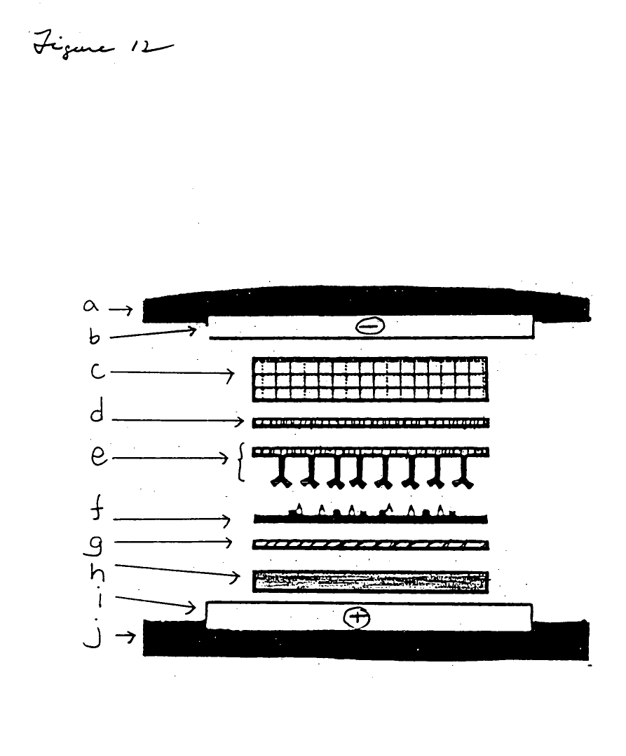

Voltage also depends on the membrane in use, and for an apparatus of the type shown in FIGS. 1 and 2 with the cathode extended part way into each well of a standard filter bottom microtiter plate, and the anode separated from the filter bottom of the plate only by a sheet of filter paper, a voltage of about 25 volts is applied when using charged nylon membranes and about 7 volts to about 8 volts when the membrane is PDVF. Preferred voltage gradients thus range from 15 to 40 volts per cm for charged filters, particularly nylon, and from 5 to 10 volts per cm for neutral filters such as PDVF.

Typically, electrophoresis is run for 5-6 minutes at 1 mA per well when conducted in a standard 96 well microtiter plate.

In addition to static fields and constant current, among the preferred embodiments of this aspect of the invention are switching fields. Indeed, in some circumstances using a pulsed electrical field can increase the sensitivity of ELISAs and ELISA-like assays about 10 fold over the use of constant voltage or constant current regulation (and the like). For instance, using a Hoeffer pulse generator, switching the electric field back and forth at a ratio 3 forward to 1 reverse and using a pulse rate of 10 msec to 50 msec substantially accelerates antibody-antigen reactions. Likewise, other ELISA reactions in which the forward to reverse polarity time ratio was 2:1, the intervals were 0.1 sec and field times were 10 msec for reverse polarity and 20 msec for forward polarity, substantially improved performance was observed in comparison to constant current regulation.

Coating

Electrophoresis can be used for membrane capture; but, other methods may be advantageous for this purpose. However, when the electrophoresis method was formally tested for capture versus weak vacuum, the vacuum method gave superior results in a standard ELISA (see FIG. 4). Speculatively, the use of a vacuum maximizes exposure of the antibody to the porous, but hydrophobic charged membrane, by pulling the antibody through more slowly than electrophoresis. Also, the forces involved in membrane capture are different than those involved in stable antigen-antibody capture and may not be improved by electrolytic activity. Therefore, weak vacuum is used for applying the antigen or antibody to the membrane in an E3 Coating Buffer.

Devices

In one of its aspects certain of the preferred embodiments of the invention herein described provide devices for electrophoretically enhanced assays. Particularly preferred embodiments in this regard provide devices designed to carry out electrophoretically enhanced analyte determinations, as described in detail elsewhere herein, especially qualitative determinations of the absence and/or the presence of a target analyte, and particularly especially quantitative determinations of the amount of the target analyte, such as its concentration and/or density and/or flux and/or amount in a sample and/or in a portion of a sample, such as in an aliquot of a fluid sample, and including both relative measurements using a reference standard for quantitation and/or absolute quantitative measurements of the amount of analyte in the sample.

Among the very most highly particularly preferred embodiments in this regard are devices in accordance with the invention that allow electrophoretically enhanced assays to be carried out in standard multiwell plates, including in certain of the particularly preferred embodiments, plates of the densities commonly employed in high throughput screening assays, in biomedical research internationally, and in clinical laboratory tests, including, for instance, in particular, 384 well plates and 96 micro well plates, and in less commonly used plates as well, such as higher and lower density plates.

In this regard the invention provides devices of the type exemplified by the device illustrated in FIG. 1, which was designed particularly for use with 96 well microtiter plates, and which allows for electrophoresis within the wells of 96 well plates. Devices in accordance with the present invention, such as those of FIG. 2, together with certain buffers provided by the present invention in another of its aspects, provide significant reductions in assay times over conventional methods and, in addition, provide a variety of performance improvements.

Illustrative of devices in accordance with various aspects and preferred embodiments of the invention, the following device was made. The device was designed particularly for E3. The device was made from light weight, highly wear resistant and low friction material, in this case Delrin, a well known plastic made by DuPont that is, in addition to the foregoing, easy to machine. Delrin provides high volume resistivity (1×10¹⁴ Ohm/cm), low water absorption (less than 0.25% in 24 hours), and good tensile strength (10,000 pounds per square inch). It is available in sheets and can be machined to shape. The apparatus described in this example is schematically depicted in FIG. 1.

The electrodes in the device illustrated in FIG. 1 can be gold plated. However, gold plating is not as resistant to wear as might be desired in some circumstances and applications, in which case, alternative materials that are more resistant to wear are preferred, such as titanium and platinum. The latter provides very high resistance to corrosion and wear; but, it is prohibitively expensive for many applications.

The upper block and the lower block of the apparatus illustrated in FIG. 1 are clamped together by four nylon screws. The screws can be inconvenient, however, and can fracture as well. Where the weight of the upper unit is sufficient to keep the upper and lower blocks properly together, as is the case for the apparatus illustrated in FIG. 1, screws and other clamping devices for keeping the two blocks together are unnecessary and may be omitted. This would make it easy to open the apparatus without turning off the power to the electrodes. Thus, where a positive clamping mechanism is not employed, for safety reasons, it is preferable for the device to incorporate male and female power connections disposed so that removing the upper unit or otherwise separating the two blocks of the device automatically and necessarily disconnects the power source.

Electrophoretically Enhanced ELISAs (E3)

The invention provides in one important aspect in accordance with the disclosure herein set forth, methods for forming complexes highly specific complexes of various types much more rapidly then is possible with conventional methods. Moreover, complexes formed in accordance with the present invention are more stable than those formed by conventional methods. This promotion of antigen/antibody immune complex formation allows a typical capture ELISA to be completed in as little as 15 minutes.

The E3 rapid ELISA format to be described here is applicable to DNA, RNA and protein, and other antigenic molecules, e.g. carbohydrates. The assay offers significant advantage to standard plate ELISA without compromising on the familiar benefits that the standard ELISA provides.

The invention thus provides, among other things, assays that: are extremely rapid compared to conventional assays, have low levels of background noise and are more sensitive than comparable assays, are quantitative over a wider range than conventional assays, can be used easily even with complex matrices, in particular those that have been difficult to handle with conventional methods, such as serum samples and cell lysates, are highly reproducible and can readily be standardized, are relatively easy to automate, can be used to detect multiple analytes in a single well, require less reagent per assay than comparable conventional assays, readily can be carried out using many conventional, standard techniques and reagents, and lend themselves to novel applications beyond the capabilities of conventional assays.

The principle of this E3 assay is the use of free solution electrophoresis to rapidly move molecules and more importantly to promote stable immunological complex formation.

A typical procedure for ELISA is described below for an immunoassay of an antigen using a primary antibody that specifically binds the antigen and a secondary antibody conjugated to an enzyme to detect the complex of the primary antibody bound specifically to the antigen. The time required to carry out the procedure conventionally is summarized and contrasted with the considerable time savings afforded by carrying out the same assay using E3.

Coating

Antigen or capture antibody is fixed to the plate by applying each in a coating buffer at pH 9.5 and incubating for 60 minutes up to overnight. High pH facilitates the binding of proteins to the plastic surface.

Detection

After the unbound antigen or capture antibody is washed away, to achieve adequate signal to noise ratios generally it is desirable and frequently it is necessary to undertake measures to minimize non-specific binding of reagents that can lead to the production of spurious signals and high background signal in the absence of the antigen. Generally, therefore, sites of non-specific binding are saturated with unlabeled substances with binding properties akin to those of the noise-generating reagents used in the assay. In ELISAs directed to biological analytes the reagents generally are polypeptides and proteins, such as a secondary antibody-enzyme conjugate of the type described in various examples elsewhere herein. Accordingly, sites of non-specific binding are blocked by incubating the wells with a highly concentrated neutral solution containing one or more “inert” proteins, e.g., albumin, casein or gelatin. The concentration of protein should be sufficiently high both to provide protein vastly in excess over sites of non-specific binding and to “drive” the non-specific binding reaction, which in part is weak and reversible, as far as is practical toward full occupancy by the blocking proteins. Blocking proteins likewise desirably do not bind to the target analyte at all so that the net result is saturation of non-specific binding sites, while leaving available the sites of specific antigen-antibody binding. In conventional ELISAs blocking typically is carried out by incubating the wells with blocking solution for 60 minutes.

Binding Reaction I

(A) Indirect ELISA: primary antibody that can bind to the fixed antigen is applied. The binding is performed in a neutral pH buffer containing excess blocking proteins to promote specific binding of the antibody to the antigen. Incubation is typically for an hour to overnight.

(B) Capture ELISA: antigen that can bind to the fixed capture antibody is applied. Again the neutral pH buffer with blocking proteins is used. Incubation is typically for an hour to overnight.

Binding Reaction II

After the unbound primary antibodies or antigens are washed away, enzyme-linked secondary or detection antibodies are applied. Incubations are usually 30 to 60 minutes.

Signal-Generating Substrate

After the unbound enzyme-linked secondary antibody or detection antibody is washed away, substrate is added. Substrate may develop a color change, or emit photons, or fluorescence.

Detection

Apparatus to quantitate the end product of the substrate/enzyme reaction is used, e.g. calorimetric, fluorescent, chemiluminescent plate reader. In general, electrophoretically enhanced reactions can utilize without modification the same detection systems used by comparable reactions carried out without enhancement. Detectable labels and measurements for use in the invention in this regard thus include those of conventional methods including colorigenic and calorimetric methods, fluorometric methods, chemiluminescent methods, radiometric methods and the like. Preferred methods of the invention facilitate accurate quantitative analysis.

Calculating Concentrations using a Standard Curve

The concentration of an analyte detected by ELISA typically is interpolated from the relationship between signal strength and concentration for members of a fiducial series of samples containing calibrated amounts of an analyte standard. The relationship often is laid out in chart form as a plot of the known concentrations vs. the mean data values of several independent assays carried out on each of several fiducial concentrations.

Capture ELISAs generally have limited antigen binding capacities and therefore they often become saturated at relatively low antigen concentrations. As a result, ELISA standard curves typically are linear only over a short range, up to the concentration where the system becomes saturated.

As summarized in the following table, it requires about 6 hours to conduct a standard capture ELISA, while the same assay can be completed in only 40 minutes using an electrophoretically enhanced method in accordance with the present invention.

Typical Times for a Capture ELISA Procedure
Standard ELISA vs Electrophoretically Enhanced ELISA

| | Std | E3 |
|---|---|---|
| Bind Ag-Specific Ab (1) to Surface(s) | 1 hour | 10 minutes |
| Block | 1 hour | 10 minutes |
| Incubate with Samples and Controls | 1 hour | 6 minutes |
| Bind Ag-Specific Ab (2) | 1 hour | 6 minutes |
| Bind 2° Ab - Enzyme Conjugate (3) | 1 hour | 6 minutes |
| TOTAL TIME | 6 hours | 40 minutes |

Std - Standard ELISA Procedure
E3 - Electrophoretically Enhanced ELISA Procedure
Ag—Antigen
Ab—Antibody
Ab (1)—a first Ag-specific antibody
Ab (2)—a second Ag-specific antibody that can bind antigen bound to Ab(1) on a surface and that is distinguishable from Ab(1) by an Ab(2) recognizing Enzyme-Ab conjugate
2° Ab - Enzyme Conjugate (3) - a conjugate of an antibody that recognizes Ab(2) but not Ab(1) and that is conjugated to a functional reporter enzyme.
Note:
Washes are carried out between all of the steps.

Electrophoretically Enhanced Westerns (E3 Westerns)

In another aspect of the invention herein described, certain of the preferred embodiments relate to systems, devices, methods, reagents and compositions of matter, inter alia, for performing reactions on matrices, such as, for instance, in certain especially particularly preferred aspects and embodiments of the invention in this regard, Westerns. Electrophoretically enhanced Westerns in accordance with the invention in this regard provide numerous advantages and improvements over conventional Westerns, including, but by no means limited to the following: (a) accelerated reactions that provide considerable time savings over conventional methods (total time for a typical Western of just over an hour, including transfer to signal development, compared with a minimum of several hours using conventional methods); (b) simplified process steps and greater ease to use; (c) greater ease of standardization; (d) low background compared to conventional methods; (e) option for using multiple antibodies with a single membrane; (f) compatible with both calorimetric and chemiluminescent detection reagents; and (g)results in the rapid formation of remarkably stable and highly specific binding complexes compared with the speed of formation and stability of complexes formed by conventional methods, especially, in particular, immune complexes of the type formed in Western blotting detection methods, among others.

Electrophoretically enhanced Westerns in accordance with the invention in this regard, (referred to in the following discussion as “E3 Westerns”) comprises, in certain of the preferred embodiments of one of its aspects in this respect, the application of voltage across a Western blot membrane that enhances complex formation between material on the membrane and detection reagents that, generally, are in a medium surrounding the membrane, or are in a medium contacting one side of the membrane and/or are in a matrix proximal to or adjacent to or in contact with one side of the membrane.

The voltage in preferred embodiments of the invention in this regard is preferably a varying voltage, particularly a pulsing voltage, especially particularly a switching voltage, very especially particularly a reversing pulse switching voltage. Further in this regard, in particularly preferred embodiments the voltage varies at relatively high frequency. Further, in preferred embodiments in this regard, the voltage varies with a periodicity of 1 to 0.0001 second, preferably 0.1 to 0.001 second, particularly preferably 0.1 to 0.01 second. Still further in this regard, preferred voltages are approximately square wave pulses of duration of 1 to 0.0001 second, preferably 0.1 to 0.001 second, particularly preferably 0.1 to 0.01 second. In yet still further preferred embodiments of the invention in this regard, preferred approximately square wave pulses of the foregoing preferred durations are applied with forward polarity and are applied more of the time then pulses with reverse polarity. In particularly preferred embodiments in this regard, pulses of approximately equal duration are applied, two-thirds with forward polarity and one-third with reverse polarity.

In further particularly preferred embodiments in these and other aspects of the invention, Westerns are performed in buffers for electrophoretically enhanced Westerns, as described below, and elsewhere herein.

An apparatus for E3 Westerns in accordance with certain of the preferred embodiments of the invention in this regard comprises, in general, electrodes configured to apply voltage across a membrane so as to electrophoretically enhance, inter alia, complex formation between substances attached to the membrane and reagents in solution. Generally, in accordance with the invention the reagents are in solution in contact with the membrane and the electrodes are in conductive communication with the solution, with at least one of the electrodes so disposed on one side of the membrane, and at least one other electrode so disposed on the other side of the membrane. The electrodes generally are configured to apply voltage evenly across the surfaces of the membrane, so that the voltage drop across the membrane is similar or, preferably, substantially the same all over the membrane where electrophoretic enhancement is desired, particularly preferably wherever material has been transferred and attached to the membrane, particularly preferably wherever material has been transferred and attached to the membrane and electrophoretic enhancement is preferred. In especially particularly preferred embodiments of the invention in this regard, the electrodes are flat electrodes that substantially cover each surface of the membrane. In addition, the electrodes are configured so as to respond rapidly to changes in voltage applied by the power supply.

In particularly preferred embodiments further in this regard, the electrodes are comprised in a device for holding the electrodes, contacts for connecting the electrodes to a power supply and reservoirs for holding buffer (which are quite small in particularly preferred embodiments in this regard, for semi-dry methods of electrophoretically enhanced transfer, binding and detection). In further preferred embodiments in this regard, the electrodes are housed in a transfer and reactions apparatus, one electrode in a base and one electrode in a lid, the base and the lid so disposed that they are easily connected to house a transfer or reaction set up and allow rapid, semi-dry transfer electrophoretically enhanced Western blotting reactions.

Power supplies in accordance with the present invention in this regard provide regulated voltage, current, power and switching capacities for carrying out electrophoretically enhanced Westerns as described herein, in a wide variety of formats and conditions. The power supplies in accordance with the invention in this regard are useful for, in addition to less conventional aspects, performing E3 Westerns using formats, gels, and reagents typical of conventional Western analysis involving standard gels and membranes, immobilized antigens, such as polypeptide antigens, and detection reagents including but by no means limited to antibodies, antibody derivatives, antibody conjugates, antibody-binding reagents, and conjugates thereof, to name just a few.

It will be appreciated that the voltage to be applied and the current generated thereby for effective electrophoretic enhancement of Westerns in accordance with various aspects and preferred embodiments of the invention herein disclosed will be a function of a variety of factors, including but not limited to the distance between the electrodes, the nature of the material disposed between the electrodes, the buffers being used, and the surface area to which the voltage is applied, to name several salient considerations, among others. Thus, a variety of power supplies may find use in E3 Westerns in accordance with the present invention.

Power supplies for use in a given circumstance in accordance with the invention in this regard preferably provide voltage, current, power and switching capability effective for electrophoretic enhancement of Westerns, including acceleration of binding reactions, enhanced formation of tight complexes, optionally reverse voltage background reduction and further optionally reverse voltage stripping of binding reagents.

In particular in accordance with the invention herein disclosed relating in particular to E3 Westerns, power supplies for use in E3 Westerns are switching power supplies that have the capacity to apply and regulate an electromotive force (“emf”) electrophoretic enhancement of reactions in accordance with various aspects and preferred embodiments of the invention. Especially, preferred power supplied for electrophoretic enhancement in accordance with the invention are switching power supplies, by which is meant that the power supplied has the capacity to supply a regulated changing voltage, in particular a pulsed voltage. Furthermore, the power supplies in preferred aspects and embodiments of the invention also can reverse polarity. In other words, power supplies in accordance with preferred aspects and embodiments of the invention have the capacity to provide a regulated, temporally changing voltage with the capacity to reverse polarity of the voltage during the program of temporal variation.

In particularly preferred aspects and embodiments of the invention in this regard, preferred power supplies supply approximately square wave voltage pulses of user defined amplitude (typically voltage), polarity, duration and frequency. A typical pulse program for E3 Westerns in accordance with this aspect of the invention is a pulse frequency of 100/sec, pulse duration of about 10 milliseconds, an amplitude per pulse of about 10 volts (see discussion below regarding voltages for E3 Westerns), with two of every three pulses with forward polarity and one of every three pulses with backward polarity.

In particular, in accordance with preferred embodiments of certain aspects of the invention, power supplies for electrophoretically enhancing reactions, including assays, such as, in particular ELISAs and Westerns (e.g. E3 ELISAs and E3 Westerns), among others, provide temporarily alternating fields, such as but not limited to pulsed alternating fields and polarities. In particular in this regard, alternating fields in accordance with certain preferred aspects of the invention in this regard are substantially square wave pulses with alternating polarity. In preferred embodiments of further aspects of the invention in this regard, there are more forward polarity pulses than backward polarity pulses.

In this regard, it is important to note that a constant electromotive force used for electrophoretic enhancement, while providing an increment of improvement over non-electrophoretically enhanced methods, does not provide as much enhancement as does a net zero alternating electromotive force (i.e., the integral sum of total applied forward and reverse electromotive force is zero), and the best results are achieved by a combination of alternating and net forward electromotive force.

By forward is meant in a desired direction and by reverse is meant in the opposite direction. For instance, an applied voltage that desirably attracts reagents towards a membrane would be a forward polarity, while opposite polarity, which would attract the same reagents away from the membrane would be the reverse polarity in this case. It will be appreciated that the terms are used relative to a given situation, and that in one situation the polarity properly designated as forward may, in the exact same setting—but at a later point in a procedure—be properly designated the reverse polarity. For instance, forward for reacting a reagent in a filter with antigens immobilized on a membrane is the polarity that attracts the reagent from the filter towards, onto and/or into the membrane, where it can interact with the antigen. The reverse then would be the polarity that attracts the reagent away from the membrane. Later in the procedure, a cleaning step may be carried out to remove weakly bound substances from the same membrane, following the formation of tight complexes between the reagent and the immobilized antigens. In this step forward it the direction that removes the non-specifically bound material and, hence, is the polarity's that would attract the reagent away from the membrane. Reverse, at this point, is the polarity that attracts the reagent to the membranes. In other words, forward designates the desired direction of migration and the polarity appertaining thereto. Reverse designates the opposite direction and polarity.

Without limiting the invention to any one mechanism or understanding of underlying processes, it is worth noting that electrophoretic enhancement in accordance with various aspects and preferred embodiments of the invention involves the application of an electric field of varying nature to cause motion of substances in solution and/or on and/or in a matrix, including agitation (such as back and forth motion, Markov motion, and/or other periodic or aperiodic motion) thereby accelerating their interaction and reaction with other substances in solution and/or on and/or in a matrix. Additionally, the electric field also enhances the interactions between the substances themselves, in particular, accelerating and enhancing the formation of tight, stable complexes that ordinarily take considerably longer to form and/or do not form at all, potentially by causing or accelerating or amplifying intramolecular motions of the substances.

Furthermore, the invention, in certain of its preferred embodiments, additionally provides for a forward electromotive force that results in a net mass flow toward a desired object, such as a net flow of reagent from solution or from a matrix to a membrane or other surface, thereby increasing the effective concentration of reactants in the vicinity of one another and, thereby, the collision frequency and, withal, the rate of the binding reaction between the reagent and cognate antigens immobilized on the membrane (for instance).

For transfer and, more especially for binding reactions in E3 Westerns in accordance with preferred embodiments of the invention in this regard, preferred power supplies have voltage capacity to provide a voltage drop across the electrodes in the range of plus and minus 1-25 volts/cm. Preferably, voltage in the range of plus and minus 2-20 volts/cm. Still more preferably plus and minus 5-15 volts/cm. Yet still more particularly preferably plus and minus 7.5-12.5 volts/cm. In the foregoing by plus and minus is meant the capacity to apply forward and reverse polarity of the indicated range of voltage drop. The distance in centimeters is the distance between the electrodes. Thus, the range of plus and minus 1-25 volts means that the power supply can apply a voltage of 1 to 25 volts per cm of separation between the electrodes, and it can apply both plus and minus to either electrode. For typical conventionally formatted E3 Westerns the distance between the electrodes typically is in the range of 1 to 2 cm and, using E3 buffers as described herein, the voltage supplied is in the range 10 to 20 volts (10 volts/cm). Note that, with switching, the extremes of the applied voltage, taking account of polarity switching are minus 20 volts and plus 20 volts; but the drop at any given time is, absolutely, 20 volts in this situation.

For transfer and, more especially for binding reactions in E3 Westerns in accordance with preferred embodiments of the invention in this regard, preferred power supplies have current capacity to provide a current of 0.1 to 10 mAmp/cm² of surface area of the electrodes, and (accordingly) of the membrane (which preferably is approximately the same in surface area as filter paper, blotting paper and the electrodes themselves (in highly preferred electrode configurations): preferably, 0.3-7 mAmp/cm²; particularly preferably 0.5-5 mAmp/cM², and especially particularly preferably 0.8-3 mAmp/cm². Yet still more especially particularly preferably is 1-2 mAmp/cm². In the foregoing mAmp means milliAmpere (0.001 Amperes), and the area in cm² is that of the surface of the electrodes between which the current is flowing and/or of the gel, membrane, filter papers and/or blotting paper through which the current is flowing. Note that the surface area referred to here is that of a flat plane parallel to the surface of the electrode, membrane, filter or blotting paper, or the like, and orthogonal (perpendicular) to the vector of current flow. Small gels often used in conventional westerns are about 10 cm by 10 cm and the surface area orthogonal to the current is 100 cm². Power supplies for E3 Westerns in this case preferably have the capacity to provide 0-500 mAmp, and would handle between 100 and 200 mAmp of current, with an applied voltage, as noted above, switching between plus and minus 12 volts, using the E3 Western buffers described elsewhere herein.

General Procedures for Electrophoretically Enhanced Westerns

Preliminary steps, prior to transfer, include, but are not necessarily limited to: gel electrophoresis of the sample(s); processing of the gel(s) prior to and in preparation for transfer (as applicable); preparation of the membrane(s) for transfer; preparation of filter paper and/or blotting paper and/or the like as needed; preparation of reagents, buffers and the like for one or more of the following, among others—transfer, washing(s), electrophoretically enhanced reaction(s), background reduction step(s), and detection step(s), set up of device(s) for transfer and electrophoretic enhancement, including the power supply(s), among others.

In many aspects E3 Westerns can employ many of the same gels, membranes, reagents and devices of conventional Westerns, except as noted herein above and in the discussion below.

Sample that can be analyzed by E3 Westerns include the same range of samples that can be analyzed by conventional Westerns. It being understood that E3 Western methods provide significant advantages and improvements over conventional methods, including much shorter assay times, formation of stronger, more stable complexes, and lower backgrounds, improved sensitivity, better and extended linearity, and increased reliability in many instances.

Gels useful for carrying out E3 Westerns include those used to carry out conventional Westerns. The gels can be any gel for separating material to be probed by western blotting methods, such as agarose, polyacrylamide and mixed agarose polyacrylamide gels, in the main the same as for conventional Westerns.

Membranes used for conventional Westerns also can be used for E3 Westerns; however, membranes should be qualified by lot for their performance in E3 Western methods, because it has been found from time to time that some lots of some types of filters perform substantially less well than expected in E3 Western methods. Among suitable membranes for E3 Westerns in accordance with the invention are PVDF, nitrocellulose and nylon membranes commonly employed in conventional blotting assays, such as conventional Westerns. Particularly preferred are PVDF membranes.

Specific substances transferred from the gel are detected on the membrane in E3 Western methods using much the same reagents as conventional Westerns. In general, as for conventional Westerns, a primary antibody (or other target-specific binding reagent or probe) is used in a first binding reaction to bind specifically to a target bound to a membrane. As discussed elsewhere in greater detail, in E3 Westerns as opposed to conventional methods the binding reaction is carried out under the influence of an electric field, preferably a pulsed field with pulses having forward and reversed polarity, in particularly preferred embodiments in a ratio of 2:1. (Details of the E3 Westerns electric field are set out elsewhere herein). Notably, this step is complete in much less time in E3 Westerns then in conventional methods for Western analysis.

It is to be appreciated that in E3 Westerns, unlike conventional Westerns, in certain aspects and preferred embodiments of the invention, the binding reagent is impregnated in a matrix—such as blotting paper—which is saturated in E3 buffer discussed below—the matrix is set onto the upper surface of the membrane (the surface that was in contact with the gel during transfer) and the reagent is migrated into contact with the membrane by both diffusion, the agitation caused by the net zero component of the polarity reversing pulse field that is applied, and by the net forward polarity component of the field which attracts the reagent from the matrix to, onto and into the membrane. As a result of the application of the field the binding reaction is accelerated and the resulting complex has unexpectedly greater stability in many cases.

Binding reagent that has not specifically complexed with the target and other non-specifically bound background-causing material is removed after the first binding reaction is complete by a washing step or steps. In conventional Westerns, the washing steps typically involve mixing and diffusion, and can be time consuming. In E3 Westerns, in some preferred embodiments, washing can be carried out conventionally; but, also may be carried out with electrophoretic enhancement and, with electrophoretic enhancement in preferred embodiments of aspects of the invention in this regard, the removal of background causing material occurs more quickly, and it is more efficient and complete than with conventional methods.

Washing typically is followed by a second binding reaction to bind a second binding reagent specifically to the target-primary antibody complex formed in the first binding reaction. Whereas this step in conventional Westerns typically is carried out by mixing and/or by diffusion, in certain preferred embodiments of the invention relating to E3 Westerns it is carried out with electrophoretic enhancement, as described in greater detail elsewhere herein. Notably, this step is complete in much less time in E3 Westerns then in conventional methods for Western analysis.

It is to be appreciated that in E3 Westerns, unlike conventional Westerns, in certain aspects and preferred embodiments of the invention, the binding reagent is impregnated in a matrix—such as blotting paper—which is saturated in E3 buffer discussed below—the matrix is set onto the upper surface of the membrane (the surface that was in contact with the gel during transfer) and the reagent is migrated into contact with the membrane by both diffusion, the agitation caused by the net zero component of the polarity reversing pulse field that is applied, and by the net forward polarity component of the field which attracts the reagent from the matrix to, onto and into the membrane. As a result of the application of the field the binding reaction is accelerated and the resulting complex has unexpectedly greater stability in many cases.

Much as for the wash described above for the wash after binding of the first binding reagent is complete, when the second binding reaction is complete, second binding reagent that does not specifically bind to the membrane bound target complex and other non-specifically bound background-causing material is removed by a wash procedure. In E3 Westerns, in some embodiments, washing can be carried out conventionally; but, also may be carried out with electrophoretic enhancement and, with electrophoretic enhancement in preferred embodiments of aspects of the invention in this regard, the removal of background-causing material occurs more quickly and is more efficiently and completely removed.

Generally, if the first binding reagent is an antibody or an antibody like or an antibody derived reagent then the second binding reagent is an anti-antibody or a protein or preparation that binds antibodies specifically, such as protein A or Staph A. Generally, either the second binding reagent itself is conjugated to a reporter moiety, such as an enzyme or a fluorophore. Otherwise, generally, a third binding reaction is carried out to bind a third binding reagent specifically to the ternary complex formed by the target, the first binding reagent and the second binding reagent. In this case, almost always the third binding reagent is specific for—generally—the second binding reagent—and comprises a reporter moiety.

For instance, in many procedures of this sort, the target is a protein antigen, the first binding reagent is a mouse monoclonal antibody specific to the antigen, the second binding reagent is either goat anti-mouse antibody conjugate to horseradish peroxidase or biotinylated goat anti-mouse antibody and, if the latter, the third binding reagent is avidin conjugated horseradish peroxidase.

Following binding of the reporter moiety containing reagent to the target complex, the membrane again is washed. Much as for the other washes described above when the binding reaction is complete, binding reagent that does not specifically bind to the membrane bound target complex and other non-specifically bound background-causing material is removed by a wash procedure. In E3 Westerns, in some embodiments, washing can be carried out conventionally; but, also may be carried out with electrophoretic enhancement and, with electrophoretic enhancement in preferred embodiments of aspects of the invention in this regard, the removal of background-causing material occurs more quickly and is more efficiently and completely removed.

After this last wash, a signal engendered by the reporter moiety is detected and from the signal intensity the amount of target bound to the membrane is calculated. This often involves comparison of the signal intensity with the signals from internal standards and—following normalization—interpolation of the amount on the membrane from a standard curve. Finally, the amount of material in the sample can be interpolated from the amount on the membrane, using standards and standard methods well known to those skilled in the art. Notably, Westerns, both conventional and E3 Westerns, can be used qualitatively as well as quantitatively, to, for instance, determine whether a target is present in a sample (rather than how much is present) and what forms of it are present.

An illustrative generalized procedure for carrying out an E3 Western in accordance with preferred embodiments of certain aspects of the invention is set out and discussed below.

Membrane, Filter Paper, Blotting Paper

Membranes, filter papers, blotting papers, sponges/blotting pads and the like should all be cut to fit the gel in advance.

Step 1—Transfer—30 minutes

Material in a gel, such as antigens and/or proteins, are transferred to a membrane. Methods of the invention in this respect PVDF membrane or other suitable membranes, such as nitrocellulose, nylon and the like, to name just a few membranes well known for their utility in various transfer and blotting assays, including but not limited to Western blotting.

In accordance with the invention herein described in certain aspects and preferred embodiments relating to E3 Westerns, transfer preferably is carried out in a semi-dry system requiring only a few milliliters of transfer buffer. However, transfer for E3 Westerns also may be accomplished by conventional methods, including wicking methods, vacuum methods and electrophoretic methods.

In accordance with the assembly for transfer shown in FIG. 11, a PVDF membrane first is wet in methanol for a minimum of 15 seconds, and then quickly washed in distilled water. Thereafter the membrane is placed in transfer buffer until used. It is important not to let the membrane dry out at any time during the procedure. Among preferred buffers for transfer for E3 Westerns is 1×E3 Transfer Buffer.

Further in accordance with the specific embodiment illustrated in FIG. 11, a piece of blotting paper is soaked in transfer buffer and then placed onto the anode of the transfer apparatus. (The membrane, the filter paper and the blotting paper all are, preferably, precut to match the dimensions of the gel.) A piece of pre-cut thin filter paper then is quickly wet in transfer buffer and placed, centered, on top of the blotting paper. It is important in placing both the blotting paper and the filter paper to eliminate any air bubbles that may be trapped between the two filter papers or between the paper and the anode, prior to initiating transfer. The pre-treated PVDF membrane (or other membrane) is placed carefully on top of the filter paper, again being careful to eliminate any air bubble between the filter paper and the membrane. The gel then is placed on top of the PVDF membrane, once again being sure to eliminate any air bubble between the gel and the membrane. A piece of filter paper cut to match the gel and soaked in transfer buffer is then placed on top of the gel, once again being careful to eliminate all air bubbles between the gel and the filter paper. A sponge, first wet in distilled water, then in transfer buffer, and pressed in the transfer buffer to expel all trapped air, is placed on top. The entire stack is shown assembled in FIG. 11.

In addition to a variety of other compositions suitable for Western transfer in conventional methods, a preferred transfer buffer for E3 Westerns in accordance with the invention is 12 mM Tris, 96 mM glycine, pH 8.7. This transfer buffer can be made up as a 25× stock: 300 mM Tris, 2.4 M Glycine, pH 8.7, and then diluted to 1× as needed. Transfer buffer preferably should be made to 15% methanol; i.e., 12 mM Tris, 96 mM glycine, pH 8.7, 15% v/v methanol. The black (−) lead from the negative terminal of the power supply is connected to the transfer apparatus, as illustrated in FIG. 11. The lid of the illustrated apparatus then is placed on top of the stack, being careful not to apply any pressure to the stack. In the illustrated apparatus of FIG. 11, the lid is kept out of contact with the base, and the weight of the lid ensures good contact throughout the stack, which, in turn provides even current flow between electrodes throughout the stack. In the assembly illustrated in FIG. 11, additional sheets of filter paper can be used to adjust the height of the stack.

The red (+) lead form the positive terminal of the power supply is then connected to the base of he illustrated apparatus. The power supply is set to maintain constant current, turned on, and set to maintain a constant current of 190 mA. Transfer is carried out for 30 minutes and the power supply is then turned off. In accordance with the assembly illustrated in FIG. 11, the power supply used for the transfer is a standard power supply, not a switching power supply. Notably, in some cases, overheating may occur with a constant current of 190 mA. If overheating occurs, the current can be decreased to about 170 mA and/or the transfer can be performed in a reduced temperature environment, such as a cold room.

At the end of the transfer the power supply is turned off. When it is certain that the power is off, the stack is disassembled carefully and the membrane is gently removed from the gel and immediately placed into blocking buffer, as described below.

Step 2—Blocking—10 Minutes

Background is reduced by incubating the membrane in a blocking buffer which, presumably, blocks sites of non-specific binding to the membrane. A variety of blocking buffers can be used for this purpose consistent with a variety of embodiments of the invention in this regard. Standard blocking buffers formulated for conventional Westerns can be used, as well as E3 Blocking Buffers formulated especially for electrophoretically enhanced methods in accordance with various aspects and preferred embodiments of the invention. A very highly particularly preferred E3 Blocking Buffer in accordance with the invention in this regard is 75 mM Tris, 450 mM glycine, 10 mM Histidine, pH 8.7, 2% milk powder. Other preferred compositions in this regard are discussed at length elsewhere herein. For membranes sized to fit gels most frequently used in Westerns, 10 mL of blocking buffer often is sufficient.

For blocking, the sponge(s) and filter papers above the membrane are removed. The membrane is carefully separated from the gel and placed face up in blocking buffer, preferably E3 Blocking Buffer as described above. (Face up in this instance means that the side of the membrane that was in contact with the gel is placed facing up in the blocking buffer). The membrane is incubated with gentle agitation in the E3 Blocking Buffer for at least 10 minutes at room temperature. Longer incubation in blocking buffer can be used, but the incubation should be carried out at 4 degrees C. if it is extended beyond one hour.

Step 3—Primary Antibody Incubation—6 Minutes

Primary antibody, specific for an antigen and/or protein of interest, is brought into contact with the membrane so as to bind specifically to any of the specific antigen and/or protein disposed thereon and/or therein. The primary antibody can be in solution or in a matrix, such as a sheet of filter paper, and preferably is in a buffer system for electrophoretic enhancement, such as the E3 buffer system described below. The incubation with primary antibody preferably is carried out using electrophoretic enhancement.

Primary antibody can be diluted in a variety of buffers. For an antibody to be impregnated into a matrix such as a filter or blotting paper, one such buffer preferred in he invention in this regard is an E3 Running Buffer, several of which are discussed elsewhere herein in considerable detail. One very highly particularly preferred E3 Running Buffer in this regard, among others, is 75 mM Tris, 450 mM glycine, 10 mM Histidine; pH 8.7.

For methods in which primary antibody will not be in a matrix and, instead will be in solution and subjected to free flow electrophoresis, a variety of loading buffers may be preferred for antibody dilution, such as the E3 loading buffers discussed in detail elsewhere herein. A very highly particularly preferred E3 Loading Buffer is 75 mM Tris, 450 mM glycine, 10 mM Histidine; pH 8.7, 2% milk powder, 20% glycerol.

Antibody incubations often are carried out in Western Running Buffer as described above and elsewhere herein. Western Running Buffer can be prepared as a 5× stock and then diluted to 1× as needed. The 5× stock should be stored at 4 degrees C. Often a volume of 100 mL in apparatus of the type illustrated in FIG. 12 is sufficient for an antibody incubation.

For incubation with primary antibody in accordance with the presently described exemplification, the transfer membrane is immersed face side up in solution containing primary antibody (i.e., the side of the membrane that was in contact with the gel is placed facing up in the solution). The membrane is incubated in the primary antibody solution with swirling for 60 seconds to ensure that the solution covers the membrane evenly. The volume of primary antibody-containing solution used for the incubation should be adjusted to the size of the membrane to provide complete and even coverage. For even coverage of full sized membranes for typically sized conventional or E3 Western gels in current use, 5 mL of primary antibody solution generally is enough.

The primary antibody-containing solution can be recovered when the incubation is complete, and stored for later use (for instance, it can be used in Step 6, below). The solution should be stored in a clean container that matches the size of the membrane as closely as possible. For E3 Westerns electrophoretically enhanced primary antibody binding in accordance with the presently described illustrative exemplification of the invention is carried out using an assembly as depicted in FIG. 12.

Just prior to removing the membrane from the primary antibody solution, a piece of blotting paper is wet in E3 Running Buffer and then centered on the anode of an E3 Western apparatus. The apparatus can be the apparatus used for transfer; but, the power supply for this step must be an E3 Western-capable switching power supply (as discussed in detail herein above and elsewhere in this disclosure). A piece of pre-cut thin filter paper wet in E3 Running Buffer is placed carefully on top of and substantially congruently with the blotting paper. It is important to eliminate all air bubbles from between the blotting paper and the anode and from between the blotting paper and the filter paper.

The antibody-incubated membrane then is placed face up on top of the filter paper. Again, it is important to eliminate air bubbles from between the membrane and the filter paper below it. A piece of pre-cut thin filter paper (pre-trimmed to the size of the membrane) wet in “Antibody Solution” from step 2 above then is placed on top of the membrane, and air bubbles are eliminated from between the membrane and the filter. The antibody soaked filter paper is covered by a pre-cut thin filter paper wet in E3 Running Buffer, once more eliminating air bubbles. The filter paper is covered with a sponge (sized to the filter paper) that has been rinsed in distilled water and then soaked in E3 Running Buffer, with squeezing to eliminate trapped air.

The leads then are connected to the power supply and to the anode and cathode of the E3 Western apparatus. The power supply is set to switching mode and turned on. The primary antibody reaction is carried out using the switching power supply for 6 minutes. The pulse program is as described for E3 Westerns herein above. At the end of the six minute power pulse reaction, the power supply is turned, the sponges and filter papers are removed and the membrane is carefully removed and washed.

Step 4—Wash—4 minutes

Wash twice in fresh PBS, two minutes each. The PBS should be Ca++ and Mg++free. Generally PBS can be prepared as a 10× stock and diluted to 1×before use. A substitute for PBS is TBS: 25 mM Tris-HCl pH 8.0, 137 mM NaCl, 2.7 mM KCl, 0.2 mM MgCl₂. TBS can be made up as a 10× stock: 250 mM Tris-HCl, pH8.0, 1.37 M NaCl, 27 mM KCl, 2 mM MgCl₂, and then diluted to 1× as needed.

Step 5—Secondary Antibody Incubation—6 Minutes

A secondary antibody or antibody conjugate, such as an enzyme-linked secondary antibody, is applied in much the same way as the primary antibody in Step 3.

Step 6—Wash—10 minutes

Wash in three changes of PBS or TBS as for Step 4. For typically sized membranes each of the three washes should be carried out for 3 minutes with gentle agitation in 20 mL of fresh PBS. If an alkaline phosphatase conjugated antibody and corresponding substrate are being used for detection the wash should be carried out in TBS instead of PBS.

Step 7—Detection—1-5 minutes

The same variety of detection methods useful for conventional Westerns can be used for E3 Westerns as well, including enzymatic, calorimetric, fluorometric, chemiluminescent, luminescent, radiographic and phosphorescent based methods, among others. Useful methods include those that involve amplification, as well as those that do not. Such methods are well known to those skilled in the pertinent arts and are not further elaborated here; but, it is to be understood that the invention is not limiting in this respect and the entire panoply of such methods available to conventional Westerns also can be employed in E3 Westerns in accordance with various aspects and preferred embodiments of the invention.

The following description of several detection methods useful in various aspects and embodiment of the invention are provided by way of illustration and are but a few of many detection systems well known to those of skill in the arts to which the invention pertains that also are useful in carrying out various aspects and embodiments of the invention pertaining to E3 Westerns, E3 ELISAs and other electrophoretically enhanced assays and processes.

Detection using a 2′ Ab-HRP Conjugate and Chemiluminescent Substrate

In this detection method a secondary antibody conjugated to horseradish peroxidase (HRP) binds to membrane-immobilized target-primary antibody complexes, and HRP conjugate bound to target complexes on the membrane are visualized by the action of the bound HRP on a chemiluminescent substrate. The HRP cleaves substrate which, as a result, generates light. The light is visualized using X-ray film.

Detection is carried out by using commercially available reagents. In particular, in the procedure of this illustrative example, 2.0 mL of chemi-substrate A is mixed with 2.0 mL of chemi-substrate B and, in accordance with the supplier's directions, the mixture is used immediately as follows.

The membrane is rinsed in 20 mL of PBS, removed from the PBS, drained briefly and excess PBS removed by contacting a corner of the membranes with a paper towel. The membrane is incubated face up for 60 seconds in fresh E-3 Western Chemi-Substrate AB solution. Following the one minute incubation, the membrane is removed from the solution, quickly drained and excess substrate is again removed by touching a corner of the membrane to a paper towel. The membrane is covered in plastic wrap and placed face up in a film cassette. A sheet of X-ray film is placed on top of the plastic-covered membrane in the cassette and kept dark for one minute. At the end of the one minute exposure, the film is removed and developed. Depending on the quality of the exposure, additional sheets of X-ray film are exposed to the membrane for longer or shorter periods of time, to obtain an optimal exposure. The amount of light from the chemiluminescent reaction product is visualized by standard methods of dosimetry, as desired.

Detection using an 2′ Ab-HRP Conjugate and a Calorimetric Substrate

The procedure for detection using a calorimetric substrate is similar to the chemiluminescent procedure described above. A secondary antibody conjugated to HRP binds to primary antibody-target complexes on the membrane. After washing, HRP bound to the immobilized target complexes on the membrane is visualized by the action of the HRP on the calorimetric substrate Tetramethyl Benzidine (“TMB”). When oxidized by HRP the substrate forms a blue precipitate on the PVDF membrane. The procedure for calorimetric detection is as follows.

The membrane is placed face up in just enough E3 Colorimetric solution to cover it evenly (about 10 ml for typical gel and membrane sizes), and incubated with the solution until the colored HRP reaction product is clearly developed, typically 10 minutes. When the reaction is complete, the membrane is removed from the solution, rinsed with distilled water, and then photographed through a yellow filter. The filter can be stored thereafter, preferably in the dark (to prevent fading).

Note on MW and Transfer in E3 Western Procedures—High MW

A number of parameters will affect the transfer of high molecular weight molecules, in an analogous way to a standard Western. Lowering the methanol concentration to 10% or less, and increasing the time of transfer can accommodate this process. However, as in a standard Western, the simultaneous transfer of both high and low molecular weight proteins using one membrane needs to be optimized, as the low molecular weight molecules will transfer at an accelerated rate and can be electrophoresed through the membrane. This is preferred to use appropriate gels (e.g. gradient gels). Prestained protein molecular weight markers can be used to estimate transfer efficiencies.

Both the foregoing disclosure and the following examples are illustrative only and not limitative. They are provided to facilitate and to ensure as true and as complete understanding of the invention as may be possible read fully by persons skilled in the arts to which it pertains. Necessarily, however, a full understanding of various aspects and applications of the invention will be possible only when viewed in the light of particular circumstances in which various of its aspects and qualities particularly pertinent thereto will come to fore and be more fully appreciated.

### EXAMPLES

### Example 1

### Device for Electrophoretically Enhanced Assays

A device for carrying out Electrophoretically Enhanced ELISA assays was made as follows. The device is illustrated schematically in FIGS. 1 and 2. It was designed to match the form factor (i.e., the size and shape) of standard 96 well microtiter plates. The device was made mainly of Delrin, a light, low friction plastic that is easy to machine to shape. Delrin is very resistant to wear, has high volume resistivity (1×10¹⁴ Ohm/cm), low water absorption (less than 0.25% in 24 hours), and good tensile strength (10,000 pounds per square inch), attractive properties for this apparatus. And it is widely available in dimensional sheets that can be worked readily and machined to shape.

Except perhaps for prototyping, other materials may be superior to Delrin. Suitable materials for the main structural parts of devices in accordance with the present invention generally should be non-conducting, wear-resistant, easy to seal, machinable, impermeable, and suitable for contact with reagents used in carrying out electrophoretically enhanced processes as described herein. Among suitable materials are acrylics, plastics, glass, plexiglass, polycarbonate, polystyrene, and other well-known materials used for electrophoresis equipment and blotting devices, among others.

The electrodes of the device depicted in FIG. 1 were gold plated, which made them somewhat more susceptible to wear then desirable. For greater wear resistance the electrodes can be made of titanium, which is highly preferred, platinum, which is costly, but highly resistant to electrochemical decomposition, palladium, which is also costly, and other materials well-known for their desirable properties in fabricating electrodes to be used in contact with ionic solutions. In a particularly preferred embodiment, where the upper electrode is a cathode, it is made of stainless steel and the anode comprises a palladium coating where it contacts the buffer (ionic solution or other conductive medium).

Since it is designed for use with 96 well plates, the unit illustrated in FIG. 1 has an upper electrode with 96 pins that extend into the wells during electrophoresis. The pins can be any shape that provides adequate contact between the running buffer and the wells and the electrode. However, the pins should not extend all the way to the bottom of the wells. Rather, they should leave enough space above the well bottoms to accommodate a layer of loading buffer covered by running buffer so that there is no contact between the upper electrode and the loading buffer. The disposition of the electrodes and the buffers in this regard is shown in FIG. 2.

### Example 2

### General Procedure for Using the Device of Example One to Carry Out Electrophoretically Enhanced Capture ELISAs

Wells of standard commercially available filter-bottom 96 well plates were coated with a capture antibody dissolved in Coating Buffer (75 mM Tris, 450 mM glycine, pH 8.7) using a well-known vacuum coating procedure. The wells were washed and then blocked with Blocking Buffer (75 mM Tris, 450 mM glycine, 10 mM Histidine, pH 8.7, 2% milk powder) to reduce non-specific binding. Blocking Buffer was removed and the wells were washed again and then partially filled with Running Buffer (75 mM Tris, 450 mM glycine, 10 mM Histidine, pH 8.7). Samples were prepared in 75 mM Tris, 450 mM glycine, 10 mM Histidine, pH 8.7, 2% milk powder, 20% glycerol (Loading Buffer) and layered into the wells beneath the Running Buffer. The lower electrode of an apparatus for carrying out electrophoretic enhancement was covered with a sheet of filter paper wetted in Running Buffer. The plate with samples then was carefully placed onto the filter paper, taking care to ensure that it was properly positioned. The lid then was closed. With the lid closed the pins of the upper electrode extended into the wells and into the Running Buffer, but not into the Loading Buffer. Voltage was applied across the electrodes to produce a current of 0.5 milliamps per well for 6 minutes, which had been shown in other experiments to be sufficient for the formation of immune complexes to be complete or nearly so. After 6 minutes the lid was opened, buffer was removed and the wells were washed. The wells were again partially filled with Running Buffer. A reporter enzyme-secondary antibody conjugate in Loading Buffer was prepared and added to the wells beneath the layer of Running Buffer. The lid was closed again, again bringing the pins of the upper electrode into the wells and into contact with the Running Buffer but not the Loading Buffer. Current was applied again for 6 minutes. By then, under these conditions, binding of the detection antibody to immobilized antigen had plateaued (i.e., was complete or nearly so). Buffers then were removed and the wells were washed. Thereafter, a standard ELISA procedure was used, without modification, to detect bound enzyme. The remaining steps thus were performed on standard equipment; although, as a matter of convenience, assays described herein generally also were completed on the E3 apparatus.

The design and operation of the device is further illustrated by the diagram in FIG. 2, which shows the disposition of the electrodes, wells and buffers in the device under running conditions. The diagram shows the lower electrode contacting the filter paper (dampened with E3 running buffer), and the filter paper lying beneath the charged hydrophobic nylon membrane. The capture antibody is immobilized on the charged hydrophobic nylon membrane that forms the bottom of the microplate and wells and traps electrophoresis products. As shown in the diagram, the hydrophobic membrane is covered by a layer of E3 Loading Buffer, which is denser than and lies underneath a layer of E3 Running Buffer. The diagram also shows the individual pins of the upper electrode plate designed to protrude individually into the wells of the plate and to extend just far enough into each well to contact the E3 Running Buffer but not the E3 Loading Buffer.

### Example 3

### E3 and Standard ELISA Procedures

Outline and comparison of typical procedures for standard ELISAs and Electrophoretically Enhanced ELISAs.

COATING

E3 Coating (60 Seconds)

The antigen or capture antibody is applied to the filter, using vacuum, in a specially formulated high pH buffer. Below is a table comparing two E3 enhanced coating procedures, one relying on vacuum coating, which is preferred, the other on electrocoating.

| E3 with vacuum coating | STEPS | | E3 with electro coating |
|---|---|---|---|
| Add 50 ml antigen to | 1 | 1 | Add 270 ml buffer to each well |
| each well | | 2 | Add 50 ml of antigen to each well as |
| | | | underlayer (carefully under the first |
| | | | buffer) |
| Incubate for 5 minutes | 2 | 3 | Add filter paper, soaked in running |
| then apply vacuum | | | buffer. |
| | | | Start electrophoresis for 6 minutes |
| Add 50 ml of blocking | 3 | 4 | Empty wells then add 270 ml |
| solution to each well | | | buffer to each well |
| | | 5 | Add 50 ml of blocking solution as |
| | | | underlayer (carefully under the first |
| | | | buffer) |
| Incubate for 5 minutes | 4 | 6 | Replace filter paper, soaked in |
| then apply vacuum | | | running buffer. |
| | | | Start electrophoresis for 6 minutes |
| Wash wells four times | 5 | 7 | Empty wells and wash four times |
| with buffer | | | with buffer |

Rest of the process is identical for both Methods

Standard ELISA Coating (30 Minutes to Overnight)

Coating is the first step and will be to fix either antigen or capture antibody to the plate by applying each in a coating buffer at pH 9.5 and incubating for 30 minutes up to overnight. High pH facilitates the binding of proteins to the plastic surface.

Blocking

Standard ELISA Blocking (30 Minutes to 2 hrs)

After the unbound antigen or capture antibody is washed away, a vast excess of inert protein (albumin, casein, gelatin, etc.) is added in a neutral pH buffer for 30 minutes up to 2 hours. This is done to block any sites where proteins could bind non-specifically, leaving available only those sites where specific antigen-antibody binding can occur.

E3 Blocking After Vacuum Coating (60 Seconds)

Unbound antigen or capture antibody is washed away using a blocking buffer applied under vacuum for 60 seconds.

Binding Reaction 1

Standard ELISA Binding Reaction I (One Hour to Overnight)

(A) Indirect ELISA: primary antibody that can bind to the fixed antigen is applied. The binding is performed in a neutral pH buffer containing excess blocking proteins to promote specific binding of the antibody to the antigen. Incubation is typically for an hour to overnight.

(B) Capture ELISA: antigen that can bind to the fixed capture antibody is applied. Again the neutral pH buffer with blocking proteins is used. Incubation is typically for an hour to overnight.

E3 Binding Reaction I (5 Minutes)

(A) Indirect ELISA: primary antibody that can bind to the fixed antigen is applied in a specially formulated buffer. Current is applied for 5 minutes.

(B) Capture ELISA: antigen that can bind to the fixed capture antibody is applied in a specially formulated buffer. Current is applied for 5 minutes.

Binding Reaction II

Standard ELISA Binding Reaction II (30 to 60 minutes)

After the unbound primary antibodies or antigens are washed away, enzyme-linked secondary or detection antibodies are applied. Incubations are usually 30 to 60 minutes.

E3 Binding Reaction II (5 Minutes)

After the unbound primary antibodies or antigens are washed away, enzyme-linked secondary or detection antibodies are applied. Current is applied for 5 minutes.

Substrate Addition

The same for E3 as for Standard ELISA. After the unbound enzyme-linked secondary antibody or detection antibody is washed away, substrate is added. Substrate may develop a color change, or emit photons, or fluorescence.

Detection

The same for E3 as for Standard ELISA. Detection: apparatus to quantitate the end product of the substrate/enzyme reaction is used, e.g. calorimetric, fluorescent, or chemiluminescent plate reader.

Calculating Concentrations Using a Standard Curve

The same for E3 as for Standard ELISA. Calculating Concentrations using a Standard Curve: A standard curve is a plot of the mean absorbance vs. the concentration of each of a series of standard samples. Because the capture ELISA has limited antigen binding capacity, the standard curve is typically linear only up to the concentration where the system becomes saturated. The assay is most accurate within the linear range.

Time

Typical times for carrying out Standard and Electrophoretically Enhanced ELISAs

### Typical Times for a Capture ELISA Procedure Standard ELISA vs Electrophoretically Enhanced ELISA

| | Std | E3 |
|---|---|---|
| Bind Ag-Specific Ab (1) to Surface(s) | 1 hour | 10 minutes |
| Block | 1 hour | 10 minutes |
| Incubate with Samples and Controls | 1 hour | 6 minutes |
| Bind Ag-Specific Ab (2) | 1 hour | 6 minutes |
| Bind 2° Ab - Enzyme Conjugate (3) | 1 hour | 6 minutes |
| TOTAL TIME | 6 hours | 40 minutes |

Std - Standard ELISA Procedure
E3 - Electrophoretically Enhanced ELISA Procedure
Ag—Antigen
Ab—Antibody
Ab (1)—a first Ag-specific antibody
Ab (2)—a second Ag-specific antibody that can bind antigen bound to Ab(1) on a urface and that is distinguishable from Ab(1) by an Ab(2) recognizing Enzyme-Ab conjugate
2° Ab - Enzyme Conjugate (3) - a conjugate of an antibody that recognizes Ab(2) but not Ab(1) and that is conjugated to a functional reporter enzyme.

Note: Washes are carried out between all of the steps

### Example 4

### Filter Coating With Vacuum or Electrophoretic Enhancement

A variety of different antigens were applied at a series of concentrations to a hydrophobic, charged nylon membrane in a 96 well pate using either a vacuum method (vac) or an electrophoretic method (E3). A standard ELISA then was used to determine the amounts of antigen that bound to the filters for each sample.

The antigens in the experiments were Epstein Barr Virus Nuclear Antigen-1 in PBS, 5% milk; IgG in ascites; glyceraldehyde-3-phosphate dehydrogenase (“G3PDH”) in mammalian cell lysate; and genomic DNA in phosphate buffer.

In the table below the data are presented as ratios. The higher results—obtained with the vacuum method—were given an arbitrary value of 100%. Results for the electrophoretic method are presented relative to 100%. The results also are presented in chart form in FIG. 4.

### Antigen Bound to Membranes

### Using Vacuum and Electrophoretic Methods

| | EBNAI | EBNAI | IgG | IgG | G3PDH | G3PDH | DNA | DNA |
|---|---|---|---|---|---|---|---|---|
| | Vac | E3 | Vac | E3 | Vac | E3 | Vac | E3 |
| 100 | 100 | 73 | 100 | 62 | 100 | 46 | 100 | 92 |
| 50 | 100 | 68 | 100 | 60 | 100 | 40 | 100 | 87 |
| 25 | 100 | 59 | 100 | 66 | 100 | 53 | 100 | 83 |
| 12.5 | 100 | 49 | 100 | 51 | 100 | 21 | 100 | 91 |

Vac - Vacuum enhanced coating and immobilization procedure
E3 - Electrophoresis enhanced coating and immobilization enhancement procedure
EBN—Epstein Barr NA1 antigen
IgG—Immunoglobulin G
G3PHD—glyceraldhyde-3-phosphate
DNA - gonomic DNA in phosphate buffer

### Example 5

### Detection of EBV-NA1

Conventional and Electrophoretically Enhanced ELISAs were carried out as described in Examples 2 and 3. Plates were coated with EBV antigen NA1 as follows. A fiducial series of known concentrations of EBNA-1 antigen was prepared in 5% milk, 95% PBS. Antigen at each dilution was bound to membranes as described elsewhere herein. Membrane bound antigen then was detected using a monoclonal anti-EBNA-1 antibody and a goat anti-mouse-antibody peroxidase conjugate. The mouse monoclonal antibody preparation was used at 1:100 dilution. The peroxidase-conjugated secondary anti-mouse antibody was used at 1:2,000 dilution. Membrane bound enzyme conjugate activity then was visualized by peroxidase activity using the chromogenic substrate TMB. Activity was quantified by densitometry of the colored reaction products on the membrane for each well using a scanning densitometry. Antigen detection was calculated from the densitometry. Results are shown in the table for two independent tests for each condition. The results are also shown in chart form in FIG. 7.

| ng/ml | ELISA | ELISA | E3 | E3 |
|---|---|---|---|---|
| 1000 | 1.482 | 1.419 | 1.984 | 1.911 |
| 250 | 1.356 | 1.321 | 2.052 | 1.987 |
| 62 | 1.104 | 1.098 | 1.821 | 1.867 |
| 15 | 0.864 | 0.871 | 1.341 | 1.239 |
| 4 | 0.578 | 0.549 | 1.003 | 0.938 |
| 1 | 0.348 | 0.329 | 0.626 | 0.589 |
| 0.25 | 0.211 | 0.231 | 0.373 | 0.358 |
| 0.062 | 0.097 | 0.107 | 0.228 | 0.241 |
| 0.001 | 0.028 | 0.037 | 0.019 | 0.008 |

ELISA - Standard ELISA
E3 - Electrophoretically Enhanced ELISA

### Example 6

### Comparison of Assay Times for an Indirect ELISA

### Standard ELISA vs Electrophoretically Enhanced ELISA

Binding end points of the ELISA described in Example 5 were compared for conventional and Electrophoretically Enhanced methods. In the conventional procedure (ELISA/10 and ELISA/60) the binding reactions for the capture antibodies and the detection antibodies were allowed to proceed either for 10 minutes each (ELISA/10) or for 60 minutes each (ELISA/60). In the electrophoretically enhanced procedure (E3) the binding reactions for the capture antibodies and the detection antibodies were allowed to proceed for 6 minutes each. Results are shown from two independent determination for each condition in the table below. The results also are shown in chart form in FIG. 8.

### Comparison of Assay Times for an Indirect ELISA Standard ELISA vs Electrophoretically Enhanced ELISA

| antigen | ELISA/ | ELISA/ | ELISA/ | ELISA/ | | |
|---|---|---|---|---|---|---|
| (ng/ml) | 10 | 10 | 60 | 60 | E3 | E3 |
| 1000 | 0.218 | 0.236 | 1.482 | 1.419 | 1.984 | 1.911 |
| 250 | 0.147 | 0.164 | 1.356 | 1.321 | 2.052 | 1.987 |
| 62 | 0.103 | 0.112 | 1.104 | 1.098 | 1.821 | 1.867 |
| 15 | 0.092 | 0.098 | 0.864 | 0.871 | 1.341 | 1.239 |
| 4 | 0.093 | 0.102 | 0.578 | 0.549 | 1.003 | 0.938 |
| 1 | 0.096 | 0.082 | 0.348 | 0.329 | 0.626 | 0.589 |
| 0.25 | 0.074 | 0.088 | 0.211 | 0.231 | 0.373 | 0.358 |
| 0.062 | 0.094 | 0.01 | 0.097 | 0.107 | 0.228 | 0.241 |
| 0 | 0.085 | 0.073 | 0.028 | 0.037 | 0.019 | 0.008 |

ELISA/10 Standard ELISA with 10 minute binding reaction for first antibody and 10 minute binding reaction for second antibody
ELISA/60 Standard ELISA with 60 minute binding reaction for first antibody and 60 minute binding reaction for second antibody
E3 Electrophoretically Enhanced ELISA with 6 minute binding reaction for first antibody and 6 minute binding reaction for second antibody

### Example 7

### Sensitivity (Limit) of Detection of an Antigen in a Cell Lysate Using an Indirect ELISA (Sandwich Assay) Standard ELISA vs Electrophoresis Enhanced ELISA

The sensitivity of conventional and Electrophoretically Enhanced ELISAs was determined for the detection of an Epstein Bar viral antigen in a cell lysate. Conventional and Electrophoretically Enhanced ELISA procedures were performed as described in Examples 2 and 3. Lysates containing the Epstein Bar Virus Viral Capsid Protein V3 (EBV VCA (V3) were prepared from a line of EBV positive B 95-8 cells in which ˜30% of the cells were VCA positive and incubated in wells of the coated plate. Plates were coated with an anti-EBV VCA (V3) capture antibody. Lysates were then incubated with the coated plate wells. A biotin-labeled anti-EBV VCA (V3) monoclonal antibody (anti-V3 clone V16) was used to detect antigen bound to the capture antibody. Streptavidin-horseradish peroxidase conjugate was used as the secondary reagent to detect membrane bound anti-V3 clone 16. Antigen detection was measured by membrane bound peroxidase activity. The peroxidase activity was quantified by measuring the amount of colored product produced by the action of peroxidase on the chromogenic substrate TMB. Following timed reaction with the TMB substrate the optical densities of colored spots on the membrane were determined using a scanning densitometer. Antigen detection was calculated from the density of color in the spots determined by the scanner. Results are set out in the table below and also depicted graphically in FIG. 6.

### Detection Limits of Cell Lysate Antigen Indirect ELISA for Standard and Electrophoresis Enhanced ELISAs

| | | | | |
|---|---|---|---|---|
| Lysate (mcg/ml) | ELISA | ELISA | E3 | E3 |

| | | | | |
|---|---|---|---|---|
| 1000 | 0.829 | 0.841 | 1.149 | 1.236 |
| 500 | 0.791 | 0.803 | 1.246 | 1.229 |
| 250 | 0.583 | 0.606 | 1.119 | 1.102 |
| 125 | 0.353 | 0.387 | 0.804 | 0.821 |
| 62.5 | 0.138 | 0.129 | 0.482 | 0.469 |
| 31.2 | 0.105 | 0.091 | 0.218 | 0.232 |
| 15.6 | 0.072 | 0.065 | 0.116 | 0.124 |
| 7.8 | 0.078 | 0.064 | 0.069 | 0.073 |
| 0 | 0.059 | 0.047 | 0.059 | 0.063 |

### Example 8

### Detection Limits in an ELISA of Modified DNA Standard ELISA vs Electrophoretically Enhanced ELISA

Conventional and Electrophoretically Enhanced ELISAs were carried out as described in Examples 2 and 3. DNA was modified with Benzo(a)pyrene diol epoxide (BPDE). Modified DNA was detected using antibody 5D11, an anti-BPDE mouse monoclonal antibody. Goat anti-mouse-peroxidase conjugate was used to detect 5D11 antibody bound to the modified DNA on the membrane. Chromogenic reactions using the TMB substrate as described in the preceding Examples were used to determine the amount of antigen detected in each well. The results obtained using standard and electrophoretically enhanced ELISAs are set out in the tables below and in chart form in FIGS. 9 and 10.

Standard ELISA

| | dsBPDE | ssBPDE | dsDNA | ssDNA |
|---|---|---|---|---|
| 1000 | 1.21 | 0.602 | 0.298 | 0.187 |
| 500 | 1.298 | 0.529 | 0.245 | 0.153 |
| 250 | 0.842 | 0.347 | 0.152 | 0.106 |
| 125 | 0.501 | 0.246 | 0.148 | 0.104 |
| 62 | 0.361 | 0.189 | 0.079 | 0.106 |
| 31 | 0.198 | 0.108 | 0.06 | 0.079 |
| 15 | 0.122 | 0.077 | 0.041 | 0.016 |
| 7 | 0.097 | 0.044 | 0.013 | 0.008 |

Electrophoresis Enhanced ELISA

| | dsBPDE | ssBPDE | dsDNA | ssDNA |
|---|---|---|---|---|
| 1000 | 2.118 | 1.071 | 0.482 | 0.271 |
| 500 | 2.002 | 0.843 | 0.322 | 0.197 |
| 250 | 1.51 | 0.71 | 0.23 | 0.151 |
| 125 | 1.184 | 0.591 | 0.169 | 0.098 |
| 62 | 0.729 | 0.31 | 0.104 | 0.078 |
| 31 | 0.353 | 0.147 | 0.073 | 0.071 |
| 15 | 0.197 | 0.103 | 0.052 | 0.032 |
| 7 | 0.103 | 0.065 | 0.028 | 0.021 |

dsBPDE—double stranded, BPDE modified DNA
ssBPDE—single stranded BPDE modified DNA
dsDNA—double stranded unmodified (control) DNA
ssDNA—single stranded unmodified (control) DNA

### Example 9

### Detection of G3PDH in Raji Cell Extracts Standard ELISA vs Electrophoretically Enhanced ELISA

Conventional ELISA was compared with Electrophoretically Enhanced ELISA in a direct assay of Glyceraldehyde-3-phosphate dehydrogenase (G3PDH) activity in Raji cell extracts. Except for the application of electrophoretic enhancement, the same standard direct ELISA protocol was used for both the conventional and the E3 assays. The determinations were carried out as follows. A G3PDH-containing mammalian cell lysate was prepared and coated onto the surfaces of wells in 96 well filter bottom plates using the vacuum coating as described above. For plastic bottom plates the lysate was incubated in the wells for two hours in accordance with standard methods in carbonate buffer. Standard ELISAs were carried out using plastic bottom plates (Styrene) or filter bottom plates (ELISA). Electrophoretically Enhanced ELISAs were carried out using filter bottom plates (E3). A polyclonal rabbit anti-G3PDH was used to detect G3PDH in membrane bound lysates. Anti-G3PDH-G3PDH were detected on membranes using an anti-rabbit IgG-peroxidase conjugate. The rabbit antibody was used at 1:100 dilution. The conjugate was used at 1:2,000. Conjugate thus bound to the membrane was determined using the chromogenic substrate TMB. Conventional and Electrophoretically Enhanced ELISAs were carried out as described in Examples 2 and 3. Results set out in the table also are presented in graphical form in FIG. 5.

Detection of G3DH in Raji Cell Extracts
Standard ELISA vs Electrophoretically Enhanced ELISA

| mcg/ml | ELISA | ELISA | E3 | E3 | Styrene | Styrene |
|---|---|---|---|---|---|---|
| 500 | 0.910 | 0.973 | 1.366 | 1.462 | 0.246 | 0.271 |
| 250 | 0.873 | 0.902 | 1.294 | 1.303 | 0.212 | 0.225 |
| 125 | 0.381 | 0.406 | 1.105 | 1.067 | 0.190 | 0.190 |
| 62 | 0.159 | 0.129 | 0.748 | 0.830 | 0.178 | 0.168 |
| 31 | 0.104 | 0.110 | 0.291 | 0.351 | 0.126 | 0.129 |
| 15 | 0.073 | 0.082 | 0.177 | 0.196 | 0.101 | 0.094 |
| 7 | 0.056 | 0.048 | 0.108 | 0.093 | 0.049 | 0.052 |
| 3.5 | 0.062 | 0.051 | 0.083 | 0.066 | 0.042 | 0.048 |
| 0 | 0.031 | 0.025 | 0.041 | 0.028 | 0.036 | 0.042 |

ELISA - Standard ELISA in filter bottom plates
E3 - Electrophoretically Enhanced ELISA in filter bottom plates
Styrene - Standard ELISA in plastic bottom plates

### Example 10

### Simultaneous Detection of Bel-X, and G3PDH (GAPDH) by E3 Western

Samples were extracted and electrophoresed through a 12% polyacrylamide gel for 90 minutes at 130 volts. Duplicate samples of each of the following samples were loaded on the gel: (1 and 2) Raji cell extract (10⁷ cells/ml, 20 microliters per lane; (3 and 4) etoposide treated Raji cell extract, 10⁷ cells/ml, 30 microliters per lane; (5 and 6) Raji cell extract (10⁷ cells/ml, 20 microliters per lane. Proteins were transferred from the gels to PVDF membranes in accordance with preferred methods for carrying out transfers for E3 Westerns, as described above. Following transfer, the procedure for incubations and washes also was as described above for E3 Westerns in general. The washed and blocked membrane was incubated with two primary antibodies at the same time: an anti-Bel-X_{L} monoclonal antibody diluted 1:1000 and a rabbit polyclonal anti-G3PDH antibody diluted 1:1000. Following the incubation with primary antibodies and washing to remove excess and non-specifically bound material, the membranes were incubated with peroxidase conjugated anti-mouse IgG and peroxidase conjugated anti-rabbit IgG (both at 1:5000 dilution). After again washing the membrane to remove further unbound and non-specifically bound material, the membrane was incubated with chemiluminescent substrate, substantially as described above. Chemiluminescence was visualized by exposing the membrane to X-ray file for 60 seconds. Thereafter, the membrane was washed with PBS and then incubated with a calorimetric substrate as provided above. At the end of the reaction the membrane was photographed.

Apotosis induced by etoposide eliminated the Bel-X_{L} signal, whereas the G3PDH signal remained the same, as expected. The results obtained by E3 Western were comparable to the best results obtained using conventional Westerns; but, the E3 results were obtained in very much less time.

### REFERENCES

The following reference whether cited in the foregoing disclosure specifically or not are herein incorporated by reference in their entirety in parts pertinent to the invention disclosure set forth herein.
1. For example see http://www.rndsystems.com/media/EDBApril02.pdf.
2. Arne W. K. Tiselius, Nobel Lecture, 1948.
3. Capillary Electrophoresis: Principles, Operations, and Applications. K. D. Altria, ed. Humana Press (1996).
4. Heller C. (2001) Electrophoresis 22: 629-43
5. http://www.interstateplastic.com/meta/fmdel.htm
6. Carswell, E. A. et al., (1975) Proc. Natl. Acad. Sci. USA 72: 3668.
7. Beutler, B. et al. (1985) Nature 316: 552.
8. Vilcek, J. and T. H. Lee (1991) J. Biol. Chem. 266: 7313.
9. Ruddle, N. H. (1992) Cuur. Opinion Immunol. 4: 327.
10. Tumor necrosis factor: structure, function and mechanism of action, Aggerwal B. B. and J. Vilcek eds., Marcek Dekker, Inc. (1991)
11. Sherry B. and A. Cerami (1988) J. Cell Biol. 107: 1269.
12. Beutler, B. and A. Cerami (1989) Annu. Rev. Immunol. 7: 625. TNF-a Immunoassay (DTA50) Instructions available at http://www.rndsystems.com
14. Ferrara N. and W. J. Henzel (1989)Biocehm. Biophys. Res. Commun. 161:851
15. Connolly, D. T. (1991) J. Cell. Biochem. 47: 219. 16. Schott, R. J. and L. A. Morrow (1993) Cardiovasc. Res. 27: 1155.
17. Ferrara, N., et al., (1992) Endocrin. Rev. 13: 1.
18. Neufeld, G., et al., (1994) Prog. Growth Factor res. 5: 89.
19. Senger, D. R., et al., (1993) Cancer and Metastatic Review 12: 303.
21. Dinarello, C. A. (1996) Blood 87: 2095.
22. Kusano, K., et al., (1998) Endocrinology 139: 1338.
23. Ling, Z. et al., (1998) Endocrinology 139: 1540.
24. Plato-Salaman, S. R. and S. E. Ilyin (1997) J. Neurosci. Res. 49: 541.
25. Hansen, M. K., et al. (1998) J. Neurosci. 18:2247.
26. Delhanty, P. J. D. (1998) Biochem. Biophys. Res. Commun. 243:269.

1. A device comprising: a first electrode, a second electrode and a reaction vessel having:(a) an outside with an outside surface; (b) an inside with an inside surface; (c) a first opening and (d) a second opening,
wherein the second opening is a semi-permeable ionically conductive membrane with an inside membrane surface that contacts the inside of the vessel and an outside membrane surface that contacts the outside of the vessel,
wherein further, during operation:
a first conductive medium is disposed in the device continuously so as to contact the first electrode, the second electrode and the outside membrane surface;
a second, conductive medium containing charged reactants or precursors thereto is disposed in the device between the inner membrane surface and the first conductive medium,
the first electrode extends through the first opening into the inside of the reaction vessel and is disposed therein in contact with the first conductive medium but not in contact with the second conductive medium
the second electrode is disposed so as to contact the first conductive medium proximal to and in conductive contact with the outside membrane surface;
wherein still further during operation voltage is applied to the first and second electrodes with net polarity that attracts the charged reactants to the membrane.

2. A device according to claim 1, comprising a first plurality of first electrodes and a second plurality of corresponding first openings, wherein during operation each first electrode of the first plurality extends through a corresponding first opening of the second plurality of corresponding first openings.

3. A device according to claim 2, further comprising a third plurality of corresponding wells, wherein during operation each first electrode of the first plurality of electrodes extends through a corresponding first opening of the second plurality of corresponding first openings and into a corresponding well of said third plurality of corresponding wells.

4. A device according to claim 3, wherein the vessel is a multiwell plate with semipermeable membrane bottom.

5. A device according to claim 1, wherein the second conductive medium is more dense than the first conductive medium and initially is disposed in a layer on top of the inside membrane surface and below the first conductive medium.

6. A device according to claim 1, wherein the semi-permeable membrane is a charged membrane or a neutral membrane.

7. A device according to claim 6, wherein the he semi-permeable membrane a nylon, charged nylon, nitrocellulose or PVDF membrane.

8. A device according to claim 7, wherein the semi-permeable membrane is a charged nylon membrane or a PVDF membrane.

9. A device according to claim 8, wherein the semi-permeable membrane is a PVDF membrane.

10. A device according to claim 1, wherein the first conductive medium is characterized by low conductivity, pH and sufficient buffering capacity for the reactants to have net charge opposite that of the second electrode.

11. A device according to claim 10 wherein the first conductive medium is an organic buffer, a weak acid or a weak base.

12 A device according to claim 11, wherein the first conductive medium is a Tris-glycine, barbital or carbonate buffer.

13. A device according to claim 1, wherein the first and second conductive media are the same except for the addition to the second conductive medium of a density increasing constituent.

14. A device according to claim 12B, wherein the first and second conductive media are the same except for the addition to the second conductive medium of a density increasing constituent.

15. A device according to claim 1, wherein a continuous voltage is applied during operation.

16. A device according to claim 1, wherein a varying voltage is applied during operation.

17. A device according to claim 16, wherein a switching voltage is applied during operation.

18. A method for carrying out reactions, comprising electrophoresis of one or more reactants in the presence of an electric field.

19. A method for carrying out reactions according to claim 18, further comprising electrophoresis is in the presence of a polarity switching electric field.

20. A method according to claim 19, wherein there is a net zero voltage component of the alternating polarity electric field that agitates a first reactant that i

21. A method according to claim 19, wherein there is a net plus or minus voltage component of the alternating polarity electric field that causes migration of a first reactant towards a surface comprising a second reactant.

22. A method according to claim 19, wherein there is both a net zero voltage component of the alternating polarity electric field that agitates a first reactant that is free in solution or in a matrix, and a net plus or minus voltage component of the alternating polarity electric field that causes migration of a first reactant towards a surface comprising a second reactant.

23. A method according to claim 22, wherein the first and second reactants are components of an immunological solid phase assay.

24. A method according to claim 23, wherein the assay is an ELISA.

25. A method according to claim 23, wherein the assay is a Western.

26. A composition for electrophoretically enhanced reactions, comprising 50-150 mM Tris Tris-glycine buffer pH 8.0 to 9.5, 300-900 mM glycine and having low conductivity.

27. A composition according to claim 26, further comprising 5 to 25 mM Histidine.

28. A composition according to claim 26, further comprising 0.5 to 2.5% milk powder.

29. A composition according to claim 26 further comprising 3%-30% density agent.

30. A composition according to claim 29, wherein the density agent is glycerol, sucrose or ficoll.

31. A composition according to claim 26, consisting in its buffering, density and blocking components essentially of 75 mM Tris, 450 mM glycine, 10 mM Histidine, 2% milk powder and 20% glycerol, and having pH 8.7.

32. A device for carrying out reactions on membrane, comprising: (a) an anode and a cathode disposed so as to a apply a voltage gradient that, when a membrane is placed between them, is even over the face of membrane and substantially perpendicular thereto, and (b) a switching power supply capable of supplying a voltage of alternating polarity.

33. A device according to claim 32, wherein the power supply has the capacity to apply a voltage that alternates polarity with a periodicity of 1 to 0.0001 second.

34. A device according to claim 33, wherein the power supply has the capacity to apply approximately square wave pulses of duration 1 to 0.0001 second.

35. A device according to claim 34, wherein the power supply has the capacity to apply the square wave pulses with forward polarity more of the time then pulses with reverse polarity.

36. A device according to claim 35, wherein the power supply has the capacity to provide alternating polarity pulses with a frequency of 100/sec, a pulse duration of about 10 milliseconds, an amplitude per pulse of about plus or minus 10 volts, and wherein two of every three pulses are of forward polarity and one of every three pulses is of reverse polarity.

37. A method for carrying out Westerns, wherein binding reaction are carried out using a device comprising: (a) an anode and a cathode disposed so as to a apply a voltage gradient that, when a membrane is placed between them, is even over the face of membrane and substantially perpendicular thereto, and (b) a switching power supply capable of supplying a voltage of alternating polarity power supply.

38. A method according to claim 37, wherein the power supply has the capacity to provide alternating polarity pulses with a frequency of 100/sec, a pulse duration of about 10 milliseconds, an amplitude per pulse of about plus or minus 10 volts, and wherein two of every three pulses are of forward polarity and one of every three pulses is of reverse polarity.

39. A method according to claim 38, wherein binding reactions are carried out in buffer comprising 75 mM Tris pH 8.7, 450 mM glycine.

40. A method according to claim 39, wherein the buffer further comprises 10 mM Histidine.

41. A kit, comprising premixed buffer composition for making a buffer comprising 75 mM Tris pH 8.7, 450 mM glycine.

42. A kit, comprising premixed buffer composition for making a buffer comprising 75 mM Tris pH 8.7, 450 mM glycine, 10 mM Histidine.
